(19) United States
(12) Patent Application Publication
(10) Pub. No.: US 2025/0281592 A1
(43) Pub. Date: Sep. 11, 2025

(54) IMMUNOGENIC COMPOSITION WITH PROTEIN MICRO- AND NANOPARTICLES

(71) Applicants: UNIVERSITAT AUTÒNOMA DE BARCELONA, BELLATERRA (ES); IRTA, INSTITUT DE RECERCA I TECNOLOGIA AGROALIMENTÀRIES, CALDES DE MONTBUI (ES)

(72) Inventors: ESTHER VÁZQUEZ GÓMEZ, EL MASNOU (ES); ANTONIO PEDRO VILLAVERDE CORRALES, EL MASNOU (ES); HÉCTOR LÓPEZ LAGUNA, RIPOLLET (ES); CARLOS MARTÍNEZ TORRÓ, BELLATERRA (ES); NATALIA MAJÒ MASFERRER, LLINARS DEL VALLÈS (ES); JORDI ARGILAGUET MARQUÈS, BARCELONA (ES); LAIA BOSCH CAMÓS, BARCELONA (ES); FERNANDO RODRÍGUEZ GONZÁLEZ, MONTGAT (ES); VIRGINIA ARAGÓN FERNÁNDEZ, TIANA (ES)

(21) Appl. No.: 18/858,944

(22) PCT Filed: Apr. 21, 2023
(86) PCT No.: PCT/EP2023/060476

§ 371 Date: Oct. 22, 2024

(30) Foreign Application Priority Data
Apr. 22, 2022 (EP) 22382383.2
Sep. 29, 2022 (EP) 22382905.2

(51) Int. Cl. A61K 39/12; A61K 39/00; A61P 37/04

(52) U.S. Cl. CPC A61K 39/12; A61P 37/04; A61K 2039/541; A61K 2039/55505; A61K 2039/55555; A61K 2039/55566; A61K 2039/55572

(57) ABSTRACT

An immunogenic composition including a particle with a particular hydrodynamic diameter, the particle comprising self-assembled protein and/or peptide molecules and one or more salts of divalent cations. The immunogenic composition can be used in various vaccines and in the prevention and/or treatment of diseases.

This application claims the benefit of European Patent Application EP22382905.2 filed on 29 Sep. 2022 and of EP22382383.2 filed on 22 Apr. 2022.

### TECHNICAL FIELD

The present invention is embedded in the field of immune system activation, including activation of innate and adaptive responses. The invention relates, thus, to the field of medicine, in particular to the field of immunogenic compositions as actives in vaccine compositions.

### BACKGROUND ART

The immune system is one of the most complex systems. Mainly, it is a network of biological processes that protects an organism from diseases by detecting and acting against pathogens, but also against cancer cells, and objects such as wood splinters, distinguishing them from the organism's own healthy tissue. Many species, including human, are provided with two subsystems of the immune system. The innate immune system, and the adaptative immune system. The first provides a preconfigured response to broad groups of situations and stimuli. The adaptive immune system tailors a response by learning to recognize molecules it has previously encountered. Both use molecules and cells from the host to perform their functions.

Cells in the innate immune system, such as dendritic cells, natural killer cells, macrophages, monocytes, neutrophils and epithelial cells recognize molecular structures produced by pathogens: pathogen-associated molecular patterns (PAMPs), which are associated with microbial pathogens; and damage-associated molecular patterns (DAMPs), which are associated with components of host's cells that are released during cell damage or cell death.

The adaptive immune system evolved in early vertebrates and allows for a stronger immune response as well as immunological memory, where each pathogen is “remembered” by a signature antigen. The adaptive immune response is antigen-specific and requires the recognition of specific “non-self” antigens during a process called antigen presentation. Antigen specificity allows for the generation of responses that are tailored to specific pathogens or pathogen-infected cells. The ability to mount these tailored responses is maintained in the body by “memory cells”. Should a pathogen infect the body more than once, these specific memory cells are used to quickly eliminate it. An adaptive immune response takes place also when the known as “self-antigens” are recognized by the immune system (i.e., autoimmune diseases) or in case of cancer vaccines against some tumoral antigens.

The cells of the adaptive immune system are the lymphocytes, being B cells and T cells the major types of lymphocytes. B cells are involved in the humoral immune response, whereas T cells are involved in cell-mediated immune response. B cells are the ones that ultimately produce and deliver to the plasma and lymph antibodies able to bind to pathogens expressing a particular antigen. This binding mark them for destruction by complement activation or for uptake and destruction by phagocytes. On the other site, T cells include among others in the so-called killer T cells or cytotoxic T cells (CD8+ cells), which only recognize antigens coupled to Class I MHC molecules, and helper T cells and regulatory T cells (CD4+ cells), which only recognize antigens coupled to Class II MHC molecules. Cytotoxic T cells (CD8+ cells) are the unique cells able to recognize an infected cell (or a cancerous cell or a foreign considered cell) and further to kill it. Helper T cells regulate both the innate and adaptive immune responses and help determine which immune responses the body makes to a particular pathogen or to a foreign-considered antigen. These helper cells have no cytotoxic activity and do not kill infected cells or clear pathogens directly. They instead control the immune response by directing other cells to perform these tasks. When B cells and T cells are activated and begin to replicate, some of their offspring become long-lived memory cells. Throughout the lifetime of an animal, these memory cells remember each specific pathogen encountered and can mount a strong response if the pathogen is detected again. This is “adaptive” because it occurs during the lifetime of an individual as an adaptation to infection with that pathogen and prepares the immune system for future challenges. Immunological memory can be in the form of either passive short-term memory or active long-term memory.

The cooperation of the innate and adaptative immunity finally aim to preserve the integrity of the individual in front of the pathogenic or antigenic foreign challenges. It is known that the type of innate immune response that is induced is going to highly influence the adaptative response and vice versa. Both responses are interconnected

Immunogens, which are substances that generates B-cell (humoral/antibody) and/or T-cell (cellular) adaptive immune responses upon exposure to a host organism can be defined as complete antigens composed of a macromolecular carrier and epitopes (determinants) that can induce immune response.

Vaccines are biological preparations that provides active acquired immunity to a particular infectious disease. A vaccine typically contains an agent that resembles a disease-causing microorganism, also called immunogen, and that is often made from weakened or killed forms of the microbe, its toxins, or one of its surface proteins. The agent stimulates the body's immune system to recognize the agent as a threat, destroy it, and to further recognize and destroy any of the microorganisms associated with that agent that it may encounter in the future. There are several types of vaccines providing different strategies to reduce the risk of illness while retaining the ability to induce a beneficial immune response. Attenuated vaccines contain live, attenuated microorganisms, which in many occasions have been cultivated under conditions that disable their danger properties. The inactivated vaccines are inactivated, but previously virulent, micro-organisms that have been destroyed with chemicals, heat, or radiation. Toxoid vaccines are made from inactivated toxic compounds that cause illness rather than the micro-organism subunit vaccine uses a fragment of it to create an immune response. The subgroup of genetic vaccines encompasses viral vector vaccines, RNA vaccines and DNA vaccines. Viral vector vaccines use a safe virus to insert pathogen genes in the body to produce specific antigens, such as surface proteins, to stimulate an immune response. An mRNA vaccine (or RNA vaccine) is a novel type of vaccine which is composed of the nucleic acid RNA, packaged within a vector such as lipid nanoparticles. DNA vaccination stems on the insertion and expression of viral or bacterial DNA in human or animal cells (enhanced by the use of electroporation), triggering immune system recognition. Some cells of the immune system that recognize the proteins expressed will mount an attack against these proteins and cells expressing them. Because these cells live for a very long time, if the pathogen that normally expresses these proteins is encountered at a later time, they will be attacked instantly by the immune system.

One of the main challenges in an immunization program is to make the antigenic determinant (i.e., the antigen) visible to all the architecture and associative components of the immune system. To this aim, many of the antigenic determinants in the vaccines are administered in combination with adjuvants, which are substances that increase, potentiate or modulate the immune response to an antigen in a vaccine. Adjuvants in immunology are often used to modify or augment the effects of a vaccine by stimulating the immune system to respond to the vaccine more vigorously, and thus providing increased immunity to a particular disease. Adjuvants accomplish this task by mimicking specific sets of evolutionarily conserved molecules, so called pathogen-associated molecular patterns, which include liposomes, lipopolysaccharide, molecular cages for antigens, components of bacterial cell walls, and endocytosed nucleic acids such as RNA, double-stranded RNA, single-stranded DNA, and unmethylated CpG dinucleotide-containing DNA. Because immune systems have evolved to recognize these specific antigenic moieties, the presence of an adjuvant in conjunction with the vaccine can greatly increase the innate immune response to the antigen by augmenting the activities of dendritic cells, lymphocytes, and macrophages by mimicking a natural infection.

It is also known that when an antigen is in some way presented in a particulate form, a better immune response is achieved than if the same antigen is solubilized or suspended in a particular media. Antigens in particulate form have distinct immunologic properties relative to soluble antigens. An understanding of the mechanisms and functional consequences of the distinct immunologic pathways engaged by these different forms of antigen is particularly relevant to the design of vaccines. It is also relevant regarding the use of therapeutic human proteins in clinical medicine that have been shown to aggregate, and perhaps as a result, elicit autoantibodies (see Snapper CM (2018) Distinct Immunologic Properties of Soluble Versus Particulate Antigens. Front. Immunol. 9:598. doi: 0.3389/fimmu.2018.00598).

However, to obtain a particle or aggregate in which the antigen is in the appropriate configuration is always a complex task, and moreover, these aggregates are still under study to elicit which are the mechanisms lying behind their action, as well as their associated drawbacks.

Examples of antigens in a somehow particulate form are disclosed in the patent document EP2755680B1 (PDS BIOTECHNOLOGY CORP), where a vaccine formulation comprising a cationic lipid adjuvant particle and a self-forming particulate protein or peptide antigen assembly is proposed. The antigen assembly comprises a micellar structure and said micellar structure is formed by attaching a hydrophobic molecule or sequence to an N-terminal amino acid residue of a protein or peptide antigen via a linker. The inventors of this document propose such a particulate formulation to boost a mammal's immune response to antigens since it includes the immunomodulator components that may advantageously accomplish one or more of the following: (1) improve antigen delivery and/or processing in the APC, (2) induce the production of immunomodulatory cytokines that favour the development of immune responses to the antigen, thus promoting cell mediated immunity, including cytotoxic T-lymphocytes (“CTL”), (3) reduce the number of immunizations or the amount of antigen required for an effective vaccine, (4) increase the biological or immunological half-life of the vaccine antigen, and (5) overcome immune tolerance to antigen by inhibiting immune suppressive factors. The boost of the immune response is further performed with the naturally or self-forming antigen assembly, such as a micelle structure or a bilayer structure, which effectively promotes larger amounts of antigen uptake by APCs compared to traditional vaccine formulations. Such an antigen assembly allows for formulation of antigens in a suitable form to be taken up and processed by APCs in a mammal, resulting in a more potent antigen-specific immune response. Furthermore, the spontaneous formation of the protein or peptide antigens into simple organized particulate structures such as micellar or bilayer structures in aqueous media allows for structures that can be effectively taken up and processed by APCs. Some of the examples in EP2755680B1 include assays with the cationic lipid R-DOTAP adjuvant nanoparticles and well-established HPV-16 E7 HLA-A2 antigenic human peptide antigens. The tested formulations contained an adjuvant of approximately 2.8 mg/ml of R-DOTAP adjuvant nanoparticles and an antigen assembly of approximately 0.83 mg/ml.

Immune responses in humanized HLA-A2 transgenic mice using vaccine formulations comprising these cationic lipid nanoparticles and antigen assemblies were evaluated by measuring induction of IFN-γ by an enzyme-linked immunosorbent spot (ELISPOT) assay. Induction of interferon-y (IFN-γ) is known to result from activated antigen-specific cytotoxic T-lymphocytes (CD8+ T-cells) and is important for development of an effective therapeutic immune response in an animal.

Most of the vaccines commercially available today in human and veterinary medicine are based on live or inactivated microorganisms. Despite their efficiency, safety concerns limit the implementation of new vaccine prototypes based on more classic methodologies. Thus, many vaccines successfully used for many decades (still in use), would have problems today to be approved by the strict regulatory offices. Therefore, implementing new vaccine formulations such as those based on subunits, has become a number one objective. So far, only a very small number of subunit vaccines have reached the market, most of them targeting very simple viruses against which, antibodies play a key protection role. On one hand, complex pathogens most probably will need complex vaccine formulations containing multiple antigens to parallel the efficacy of more classic vaccine developments. On the other hand, soluble proteins have limited ability to be presented in the major histocompatibility complex type I (MHCI), key step to induce specific CD8-Tcells cytotoxic T-cells (or CTLs), capable to specifically eliminate cells infected with intracellular microorganisms. Sterilizing protection against most intracellular pathogens require the induction of antibodies capable of recognizing and eliminating the pathogen in blood and other body fluids and CTL responses, essential to eliminate the infected cells. Conversely to soluble protein monomers, proteins formulated in nanoparticulated structures (NP), are recognized by specific subsets of dendritic cells and macrophages (professional antigen presenting cells), inducing specific CTL responses by a mechanism known as cross-presentation (ThijsW. H. Flinsenberg and Marianne Boes. Application of antigen cross-presentation research into patient care. Front. Immunol. 5:287. doi: 10.3389/fimmu.2014.00287; Muntjewerff E M, Meesters L D and van den Bogaart G (2020) Antigen Cross-Presentation by Macrophages. Front. Immunol. 11:1276. doi: 10.3389/fimmu.2020.01276).

However, in spite of all the efforts to obtain particulate vaccines, they are indeed still complex mixtures requiring of the separate components (i.e., adjuvants and antigens) that are to be processed by means of particular methods to get the effective immune response. Therefore, there is still a need for alternative or better approaches to provide effective immunogens.

### SUMMARY OF INVENTION

Inventors surprisingly found that particles formed of self-assembled proteins by means of divalent cations were able to elicit a high innate immune response as well as a high adaptative response in a model of porcine disease but also in in-vitro assays.

As will be illustrated in the examples, the administration of such particles, even without any additional adjuvant, induced the innate response when the particles had a diameter around 50 to 4000 nm, in particular around 200 to 4000 nm. This innate response was further complemented with an adaptative response (both specific B cells and T-cells were induced) when the particles, acting as secretory granules, further delivered particles of a lower size, from 5 to 150 nm, in particular from 5 to 80 nm, said nanoparticles of lower size resulting from the disintegration of the secretory granules of 50-4000 nm, or in particular of 200-4000 nm.

Interestingly, the particles of the present invention had the ability to induce not only specific antibodies (B cells) and T helper cells but also cytotoxic T cells, CTLs (CD8 T-cells).

Thus, the inventors propose the herein also called protein-only immunogenic compositions (i.e., particulate immunogenic compositions, also herein referred to as abbreviated POMV or secretion granules) applicable to any antigenic determinant (antigenic molecule) of interest capable to elicit an immune response. This is a new paradigm for the immunization of the subjects due to the POMV's capacity to induce both the innate and the adaptative immune response using particles that are, indeed, protein-only particles of self-assembled proteins or peptides in the presence of salts of divalent cations. Moreover, the immunogenic compositions proved to be non-toxic, both in vitro and in vivo.

Thus, a first aspect of the invention is an immunogenic composition comprising or consisting of a particle (i.e., granule or aggregate) with a hydrodynamic diameter from 50 to 4000 nm, said particle comprising antigenic protein and/or peptide (i.e., antigenic substances) and one or more salts of divalent cations, and wherein the antigenic protein and/or peptide remains assembled (i.e., self-assembled) in the presence of the said salts of divalent cations, being the ratio of moles of salt of divalent cation:moles of antigenic protein and/or peptide in the particle from 40:1 to 1000:1.

This immunogenic composition can also be named simply as an immunogen, and the particles constituting or being comprised in it can also be termed as secretory granules, or as protein-only particles or microparticles (“μp”) (i.e., POMV of 50 nm to 4000 nm, in particular 200 nm to 4000 nm). A schematic view of the particles or secretory granules, which secrete particles of lower size that, in turn, ultimately disintegrate in the monomeric or oligomeric integrating proteins of the particle is illustrated in FIG. 1.

In the immunogen, the particles result from the self-forming antigen assembly (i.e., protein assembly) in the presence of the divalent cations, and they are simple organized particulate structures such as micellar or bilayer structures in a polar media, such an aqueous media.

An example of these secretory granules has been previously disclosed in the international patent application with the publication number WO2020208065A1, or in the document of Lopez-Laguna et al., “In Vitro Fabrication of Microscale Secretory Granules”, Advanced Functional Materials Journal-2021, 210914, https://doi.org/10.1002/adfm.202100914. The later document discloses the potentiality of the secretory granules as functional depots for protein drugs. WO2020208065A1 discloses protein nano- or microparticles, comprising a cluster of one or more types of assembled self-contained proteins and the salts of divalent cations, the micro/nanoparticle being stable and having a size, measured as hydrodynamic diameter, from 50 nm to 50 μm. The proteins are therapeutic proteins and their granules are proposed for use in the treatment of a disease selected from the group consisting of cancer, an immune disease, neurodegenerative disease, and combinations thereof.

However, none of the two previous documents teaches or suggests the herein disclosed use of the granules (i.e., micro or nanoparticles of assembled self-contained proteins) as immunogens with the indicated and advantageous properties.

Thus, the particles or, synonymously, secretory granules as previously disclosed, with a hydrodynamic diameter from 50 to 4000 nm, more in particular from 200 to 4000 nm, said particles comprising protein and/or peptide molecules (i.e., antigenic proteins and/or peptides) and one or more salts of divalent cations in the indicated molar ratios (i.e, from 40:1 to 1000:1), are now proposed for use as immunogens or as immunogenic compositions, in particular for use in the prevention and/or treatment of diseases or disorders, more in particular caused by pathogens, and by eliciting the immune system of a subject.

The immunogenic composition or immunogen can be used as the active agent in vaccines, which are the pharmaceutical or veterinary compositions with the appropriate excipients and carriers used to immunize subjects. Thus, in a second aspect the invention includes a vaccine composition comprising a therapeutically effective amount of the immunogenic composition as defined in the first aspect, together with a pharmaceutically and acceptable excipient and/or carrier.

Indeed, both the immunogenic composition or the vaccine comprising it are capable of inducing a response by the immune system of the subject, which can further be used to prevent or treat diseases. Thus, a third aspect of the invention relates to an immunogenic composition as defined in the first aspect, or a vaccine as defined in the second aspect, for use as a medicament (i.e., for use in therapy). This aspect can be reworded as the use of an immunogenic composition as defined in the first aspect, or a vaccine as defined in the second aspect, for the preparation of a medicament. Also disclosed is a method of treatment which comprises administering to a subject in need thereof an immunogenic composition as defined in the first aspect, or a vaccine as defined in the second aspect.

A fourth aspect discloses is a particle with a hydrodynamic diameter from 50 to 4000 nm, in particular from 200 to 4000 nm, said particle comprising antigenic protein and/or peptides and one or more salts of divalent cations, and wherein the antigenic protein and/or peptides remain assembled in the presence of the said salts of divalent cations, being the ratio of moles of salt of divalent cation:moles of protein and/or peptide molecules in the particle from 40:1 to 1000:1. Further aspects disclose said particle for use as immunogen, for use as a medicament, and a vaccine composition comprising said particle.

The invention also includes, as another aspect, a method for the preparation of a vaccine as defined in the second aspect, comprising the step of mixing the immunogenic composition of the first aspect, or the particle of the fourth aspect, with a pharmaceutically acceptable excipient and/or carrier.

Finally, another aspect of the invention is a vaccination kit comprising:
(a) an immunogenic composition as defined in the first aspect, or a particle as defined in the fourth aspect;
(b) a pharmaceutically acceptable excipient and/or carrier;
(c) optionally an adjuvant; and
(d) optionally instructions for its use.

### BRIEF DESCRIPTION OF DRAWINGS

FIG. 1 is a schematic view of the particles or secretory granules, which secrete particles of lower size that in turn ultimately disintegrate in the monomeric or oligomeric integrating proteins of the particle constituting the immunogen. “In vivo” expression denotes the disintegration when the particles of the immunogen are in a physiological environment condition (i.e., in the blood stream or the extracellular fluid of a subject). “In vitro” denotes the mode the particles or secretory granules are manufactured in the presence of salts of divalent cations.

FIG. 2 shows a schematic representation of an in vivo immunization procedure with the immunogenic composition of the invention comprising the protein-only particles.

FIG. 3(A) to (H) shows the data obtained with a Luminex assay for the analysis in the supernatants of pig alveolar macrophages (PAMs) after stimulation with either GFP-POMV, which denotes the microparticles of GFP constituting the immunogen of the invention, or with the RPMI culture media (RPMI), as negative control for the assay. The cytokines measured where: tumoral necrosis factor α (TNF α), Interleukin 1b (IL-1b), Interleukin 6 (IL-6), Interleukin 12 (IL-12), interferon γ (IFN-γ), interferon α (IFN-α), Interleukin 4 (IL-4) and Interleukin 10 (IL-10). MFI stands for mean fluorescence intensity and pg/ml for pictograms of the corresponding cytokine per milliliter of PAM supernatant.

FIG. 4. African swine fever virus (ASFV)-specific immune responses induced in immunized pigs. A). Detection of ASFV-specific IgG1 and IgG2 isotypes in the blood of immunized pigs at the peak of the immune response (14 days post-boost) by ELISA. Interestingly, all pigs immunized with the POMVs (μp32), containing μp32 alone or in the presence of the CAF01, responded to the immunization, the latter showing higher averages of ASFV-specific IgG2. B) ASFV-specific T-cells detected in the inguinal lymph nodes by IFN-γ ELISPOT. Pigs immunized with either np32 or μp32 in the presence of CAF01 induced T-cells that specifically secreted IFN-γ in response to overnight stimulation with ASFV, detectable in lymph nodes at the time of sacrifice (day 28 pb), confirming for the first time, the induction of Th1-like responses after immunization with p32 (FIG. 4 (B)).

FIG. 5 shows the optical density at 450 nm (OD 450 nm) in the Y-axis, obtained by ELISA using GFP-coated plates. Results shown correspond to a serial dilution (X-axis) of the serum obtained from mice immunized with either 5 micrograms (lines grouped at the bottom of the graphics) or 50 micrograms (lines grouped at the top of the graphics, also circled with a discontinuous line) of an immunogenic composition of the invention comprising or consisting of particles (granules) of assembled GFP-H6 protein (GFP POMV). The line with empty circles at the bottom of both panels (A) and (B) corresponds to the serum of -immunized mice, negative control of the assay. The serum containing antibodies (anti-GFP-H6) was analysed by ELISA with an anti-mouse polyvalent Ig. In (A) there are the data 2 weeks after second inoculation of the particles of GFP-H6. In (B), there are the data 9 weeks after second inoculation of the particles of GFP-H6.

FIG. 6 is a graph illustrating the percentage of proliferating CD4+ T cells, and CD8+ T cells, present in the spleen from mice immunized twice with either 5 micrograms (columns 1 and 2 in the graphic reading from left to right) or 50 micrograms (columns 3 and 4 in the graphic reading from left to right) of GFP POMVs. The percentage is indicated as the detected proliferating cells, positive for the Ki37 cell-proliferation marker (% of Ki67+). Data shown corresponds to the CD8+ T-cell specific stimulation observed after 5 days of in vitro stimulation with the granules of the invention (GFP POMV). Columns five and six (on the right of the figure) are data obtained using PBMCs from non-immunized naïve mice, acting as negative control of the assay.

FIG. 7. Size of RK4-P32-H6 (A) and GFP-H6 (B) granules and their released nanoparticles as determined by Dynamic Light Scattering. The size of the released nanoparticles (soluble protein) released from the secretory granules was measured after 7 days of incubation at 37° C.

FIG. 8. FESEM micrographs of RK4-P32-H6 secretory granules. Samples were previously diluted 5× with sterile H₂O to reduce the salt concentration in order to optimize their visualization.

FIG. 9. Thermal stability of RK4-P32-H6 secretory granules measured by DLS.

FIG. 10. Example of RK4-P32-H6 secretory granules. Soluble protein was mixed with 10 mM of zinc chloride as described and, after centrifugation, a protein precipitate was formed.

FIG. 11. Release of soluble protein nanoparticles from RK4-P32-H6 secretory granules. The granules were incubated at 37° C. for a week. Samples were extracted at days 0, 1, 3 and 7 after granule formation.

FIG. 12. ASFV-specific IgG1, IgG2 (A) and IgA (B), detectable in sera from pigs immunized with two doses of 150 μg of p32-POMVS.

FIG. 13. GFP-specific IgG1, IgG2 (A) and IgA (B), detectable in sera from mice immunized with two doses of 50 μg of GFP-POMVS.

### DETAILED DESCRIPTION OF THE INVENTION

Unless defined otherwise, all technical and scientific terms used herein have the same meaning as is commonly understood by one of skill in the art to which this invention belongs at the time of filing. However, in the event of any latent ambiguity, definitions provided herein take precedent over any dictionary or extrinsic definition. Further, unless otherwise required by context, singular terms shall include pluralities and plural terms shall include the singular.

The term “antigen” refers to a molecule against which a subject can initiate an immune response, e.g., a humoral and/or cellular immune response. Depending on the intended function of the composition, one or more antigens may be included. When in this description the term “antigen” or “antigenic determinant” are used they refer to a protein, a peptide, a polysaccharide in a protein or peptide of the particles, a glycoprotein, a glycolipid in a protein or peptide of the particles, a nucleic acid in a protein or peptide of the particles, or a combination thereof.

The term “medicament” as used herein is synonymous of a pharmaceutical or veterinary drug (also referred to as medicine, medication, or simply drug) used to cure, treat, or prevent disease in animals, including humans, as widely accepted. Drugs are classified in various ways. One key distinction is between traditional small-molecule drugs, usually derived from chemical synthesis, and biopharmaceuticals, which include recombinant proteins, vaccines, blood products used therapeutically (such as IVIG), gene therapy, monoclonal antibodies and cell therapy (for instance, stem-cell therapies). In the present invention medicament preferably is a veterinary medicament, and even more preferably is a vaccine for veterinary use.

The term “vaccine” as used herein, means an immunogenic composition accompanied by adequate excipients and/or carriers, that when administered to an animal, elicits, or is able to elicit, directly or indirectly, an immune response in the animal. Particularly, the vaccines of the present invention elicit an immunological response in the host of a cellular or antibody-mediated type upon administration to the subject that it is protective. The vaccine includes as “active principle” an “immunogenic composition”, which as it is used herein, refers to a material that elicits an immunological response in the host of a cellular or antibody-mediated immune response type to the composition upon administration to a vertebrate, including humans. The immunogenic composition comprises molecules with antigenic properties, such as killed or attenuated bacteria or virus, among others, and also immunogenic polypeptides (proteins or peptides). An immunogenic polypeptide is generally referred to as antigenic. A molecule is “antigenic” when it is capable of specifically interacting with an antigen recognition molecule of the immune system, such as an immunoglobulin (antibody) or T cell antigen receptor. An antigenic polypeptide contains an epitope of at least about five, and particularly at least about 10, at least 15, at least 20 or at least 50 amino acids. An antigenic portion of a polypeptide, also referred to as an epitope, can be that portion that is immunodominant for antibody or T cell receptor recognition, or it can be a portion used to generate an antibody to the molecule by conjugating the antigenic portion to a carrier polypeptide for immunization.

As for the expression “immunologically effective amount,” or “immunologically effective dose” means the administration of that amount or dose of antigen, either in a single dose or as part of a series, that elicits, or is able to elicit, an immune response that reduces the incidence of or lessens the severity of infection or incident of disease in an animal for either the treatment or prevention of disease. The immunologically effective amount or effective dose is also able for inducing the production of antibody for either the treatment or prevention of disease. This amount will vary depending upon a variety of factors, including the physical condition of the subject, and can be readily determined by someone of skill in the art.

The expression “remain assembled” when referring to the proteins and peptide molecules in the particles comprised or constituting the immunogenic composition, or equivalently, “self-assembled proteins” relates to protein clusters of protein or peptide molecules defining discrete groups or bodies, such as particles.

As used herein, the indefinite articles “a” and “an” are synonymous with “at least one” or “one or more.” Unless indicated otherwise, definite articles used herein, such as “the” also include the plural of the noun.

The expression “therapeutically effective amount” as used herein, refers to the amount of a compound that, when administered, is sufficient to prevent development of, or alleviate to some extent, one or more of the symptoms of the disease which is addressed. The particular dose of compound administered according to this invention will of course be determined by the particular circumstances surrounding the case, including the compound administered, the route of administration, the particular condition being treated, and the similar considerations.

The expression “pharmaceutically acceptable excipients or carriers” refers to pharmaceutically acceptable materials, compositions or vehicles. Each component must be pharmaceutically acceptable in the sense of being compatible with the other ingredients of the pharmaceutical composition. It must also be suitable for use in contact with the tissue or organ of humans and animals without excessive toxicity, irritation, allergic response, immunogenicity or other problems or complications commensurate with a reasonable benefit/risk ratio. The particular dose of compound administered according to this disclosure will of course be determined by the particular circumstances surrounding the case, including the compound administered, the route of administration, the particular condition being treated, and similar considerations. In one embodiment, the amount of active ingredient administered (or dose) may be from about 0.5 micrograms/kilogram body weight to about 40 milligrams/kilogram body weight per day In another embodiment, the dose may be from about 0.1 micrograms/kilogram body weight to about 10 milligrams/kilogram body weight per day. In another embodiment, the dose may be from about 0.5 micrograms/kilogram body weight to about 0.1 milligrams/kilogram body weight per day. In another embodiment, the dose may be from about 10 micrograms/kilogram body weight to about 40 milligrams/kilogram body weight per day. In another embodiment, the dose may be from about 0.1 micrograms/kilogram body weight to about 10 milligrams/kilogram body weight per day.

As above indicated, a first aspect of the invention is an immunogenic composition comprising or consisting of a particle with a hydrodynamic diameter from 50 to 4000 nm, said particle comprising protein and/or peptide molecules and one or more salts of divalent cations, and wherein the protein and/or peptide molecules remain assembled (i.e., self-assembled) in the presence of the said salts of divalent cations, being the ratio of moles of salt of divalent cation:moles of protein and/or peptide molecules in the particle from 40:1 to 1000:1.

In a particular embodiment, the immunogenic composition comprises or consists of a particle with a hydrodynamic diameter from 200 nm to 4000 nm. In another particular embodiment, the immunogenic composition comprises or consists of a particle with a hydrodynamic diameter from 200 nm to 3000 nm. In another particular embodiment, the immunogenic composition comprises or consists of a particle with a hydrodynamic diameter from 200 nm to 2000 nm.

In a particular embodiment of the first aspect, the particle has a release profile under physiologic conditions in phosphate buffered saline and neutral pH, preferably without agitation in which an amount from 30% to 100% by weight in relation to the total weight of the antigenic proteins and/or peptides is released in form of nanoparticles with a hydrodynamic diameter from 5 to 80 nm. If agitation is needed, gentle agitation (preferably below 30 rpm) may be applied. In a particular embodiment of the first aspect, the particle has a release profile under physiologic conditions in phosphate buffered saline and neutral pH, in which an amount from 30% to 100% by weight in relation to the total weight of the proteins and/or peptide is released in form of nanoparticles with a hydrodynamic diameter from 5 to 50 nm, more in particular from 5 to 15 nm with a time from 24 h to 10 days. In a more particular embodiment, the time in which said 30-100% of antigenic proteins and/or peptides is released in the form of nanoparticles is from 24 h to 7 days.

The release is, in another particular embodiment, from 20% to 80% within a time of 24-48 h and under the previous conditions. In another alternative particular embodiment, the release is from 20 to 80% by weight in relation to the total weight of the proteins and/or peptide molecules within 2 weeks.

Physiological conditions include a temperature from 34.5° C. to 42° C. (being normal from 36.5° C. to 37.5° C. (the said physiological temperature) and pH around 7 (6.5-7.8). The release is also to be understood as a mode of delivering said proteins from the particles that disintegrate in a particular media. The media can be, in particular, a tissue from a living organism in such a way that the particle is finally decomposed, in particular in a sustained way.

For the assay of the release, the particle is submitted to the indicated physiological conditions, preferably without agitation. In general the conditions simulate the conditions in the body (pH, liquid media, and circulation).

In the same way, the measure of the delivered particles of small size in relation to the particle from which they are segregated is, in a particular embodiment measured by immunoassays. This way the percentage of release can be calculated. More in particular this release has been observed in vitro by recovering the supernatant and measuring the amount and size of the segregated nanoparticles (i.e., from 5 to 80 nm). The said release can also be measured in vivo by measuring the amount and type of protein retained in an implant and the one that is accumulated to a targeted tissue, such as a tumour.

In a more particular embodiment of the immunogenic composition, the salts of divalent cations include single and multiple salts (i.e., double salts), and combinations thereof. In a more particular embodiment, the divalent cations of the salts are selected from the group consisting of Be²⁺, Mg²⁺, Mn²⁺, Ca²⁺, Sr²⁺, Ba²⁺, Ra²⁺, Zn²⁺, Cu²⁺, Ni²⁺, and combinations thereof. In a particular embodiment, divalent cations of the salts are alkaline-earth cations. The salts are, in particular embodiment inorganic salts of divalent cations, more in particular of Be²⁺, Mg²⁺, Mn²⁺, Ca²⁺, Sr²⁺, Ba²⁺, Ra²⁺, Zn²⁺, Cu²⁺, Ni²⁺, and combinations thereof. Particular salts include CaCl₂, ZnCl₂, NiCl₂, and combinations thereof. More in particular, the salt is a Zn²⁺ salt. Even more in particular, the salt is ZnCl₂.

In another particular embodiment of the first aspect, optionally in combination with any embodiment above or below, the assembled proteins and/or peptide molecules comprise one or more amino acids that due to the presence of charge at physiological pH and/or of the presence of aromatic or heteroaromatic structures can coordinate with the divalent cations. This coordination controls the self-assembly of the structure (i.e., of the proteins/peptides in presence of salts of divalent cations). In another particular embodiment of the first aspect, optionally in combination with any embodiment above or below, the assembled proteins and/or peptide molecules comprise one or more histidine residues. Thus, they are histidine-containing proteins or peptides.

If not being antigens with naturally occurring histidines in their sequences, the assembled protein or peptide molecules in the particle comprised in or consisting in the immunogenic composition are artificially provided with histidine sequences at any of their N- or C-terminal ends. As previously indicated, this aims to coordinate the divalent cations of the salts and then to promote the self-assembly of the protein/peptides to form the discrete particles. In another particular embodiment, optionally in combination with any embodiment above or below, the protein and/or peptide molecules in the particle comprise a polyhistidine-tag, thus besides being antigenic proteins they contain several histidines in the sequence (i.e., they are His-tagged proteins). His tagged proteins are also known as histidine-rich proteins, which are proteins, usually recombinant proteins, comprising a polyhistidine-tag. His-tagged proteins according to present invention also include proteins with a number of histidines in their amino acid sequence selected from 3, 4, 5, 6, 7, 7, 9 and 10 histidines. The polyhistidine-tag is an amino acid motif in proteins that usually consists of at least six histidine (His) residues, often at the N- or C-terminus of the protein. Some proteins also comprise these at least six histidine residues in the middle of their amino acid sequence, such as in loop regions. This motif of histidine residues is also known as hexa histidine-tag, 6×His-tag, His6 tag and by the trademarked name His-tag (registered by EMD Biosciences). His-tagged proteins according to present invention also include natural proteins that comprise high amounts of histidine amino acid in their sequences.

Thus, in another particular embodiment of the first aspect, optionally in combination with any of the embodiments above or below, the immunogen comprises or consists in particles (i.e., granules or aggregate) with a hydrodynamic diameter from 50 to 4000 nm, in particular from 200 to 4000 nm, more particularly from 200 to 2000, said particles comprising:
antigenic protein and/or peptides with one or more amino acids that due to the presence of charge at physiological pH and/or of the presence of aromatic or heteroaromatic structures can coordinate with the divalent cations, in particular comprising polyhistidine-tags; and
salts of divalent cations;
and wherein the protein and/or peptide molecules remain assembled (i.e., self-assembled) in the presence of the said salts of divalent cations, being the ratio of moles of salt of divalent cation:moles of protein and/or peptide molecules in the particle comprised from 40:1 to 1000:1.

Depending on the selected protein and/or peptide molecules that is forming part of the immunogenic composition, the one or more amino acids that coordinate with divalent cations can differ. In the particular embodiment, the said protein and/or peptide molecules comprise added tags of such amino acids that coordinate with the divalent cations, selected from aromatic or heteroaromatic amino acids, in particular, they comprise polyhistidine tags. These tags are, as the skilled person in the art will understand, added synthetically or by protein recombinant technologies of common practice. In another particular embodiment, the proteins and/or peptide molecules comprise a cationic terminal domain, in particular a cationic N-terminal domain. This cationic domain may be part of the original protein and/or peptide molecule or it may be added synthetically or recombinantly to the original protein and/or peptide molecule. More particularly, in some embodiments the cationic terminal domain comprises arginine and/or lysine residues.

In another particular embodiment of the immunogenic composition of the first aspect, optionally in combination with any of the embodiments above or below, the proteins and/or peptide molecules are selected from one or more antigenic proteins and/or peptides selected from the group consisting of a viral antigenic protein or peptide, a bacterial antigenic protein or peptide, a fungal antigenic protein or peptide, a protozoa or parasite antigenic protein or peptide, a cancer antigen, toxin antigen, venom antigen, autoimmune causing disease antigen (or synonymously self-antigen), allergenic antigen, and a pathogenic antigen.

In various embodiments described herein, the antigen or protein/peptide assembly comprises one or more antigens of different amino acid sequences, thus, they are not identical proteins and/or peptides.

In another more particular embodiment, the proteins and/or peptide molecules are selected from one or more antigenic proteins and/or peptides selected from the group consisting of a viral antigenic protein or peptide, and a bacterial antigenic protein or peptide, a parasitic antigenic protein or peptide.

Even in another more particular embodiment, the viral antigenic protein or peptide is one of an animal infecting virus, the bacterial antigenic protein or peptide is one of an animal infecting bacteria, and the parasitic antigenic protein or peptide is one of an animal infecting parasite. More in particular, the infecting virus, bacteria, or parasite is a mammal infecting virus, bacteria or parasite.

Particular mammal infecting virus, bacteria, or parasite are those causing diseases in human; in livestock, such as swine, cows, poultry; and in domestic animals, such as dogs, cats, and horses.

As will be illustrated in the examples with a challenge of African swine fever virus, in another particular embodiment of the immunogenic composition, the viral antigenic protein comprises protein p32 of African swine fever virus or a fragment thereof.

In one embodiment, the pathogenic antigen is a synthetic or recombinant antigen. In another embodiment, the pathogenic antigen is an isolated bacterial protein.

Non-limiting bacteria from which the antigenic proteins or peptides may originate include, Aceinetobacter calcoaceticus, Acetobacter paseruianus, Actinobacilius actinomycetemcomitans, Actinobacilius pleuropneumoniae, Actinomyces israelii, Actinomyces viscosus, Aeromonas hydrophila, Alcaliges eutrophus, Alicyclobacillus acidocaldarius, Arhaeglobus fulgidus, Bacillus species, Bacillus antracis, Bacillus pumilus, Bacillus stearothermophillus, Bacillus subtilis, Bacillus thermocatenulatus, Bacteroides species, Bordetella species, Bordetella bronchiseptica, Borrelia burgdorferi, Brucella species, Burkholderia urvivin, Burkholderia glumae, Brachyspira species. Brachyspira hyodysenteria, Brachyspira pilosicoli, Camphylobacter species, Campylobacter coll, Campylobacter fetus, Campylobacter hyointestinalis, Campylobacter jejuni, Chlamydia psittaci, Chlamydia trachomatis, Chlamydophila species, Chromobactehum viscosum, Clostridium species, Clostridium botulinum, Clostridium difficile, Clostridium perfringens, Clostridium tetani, Corynebacterium species, Corynebacterium diphtheriae, Ehrlichia canis, Enterobacter species, Enterobacter aerogenes, Enterococcus species, Erysipelothrix rhusiopathieae, Escherichia species, Escherichia coli, Fusobacterium nucleatum, Haemophilus species, Haemophilus influenzae, Haemophilus somnus, Helicobacter species, Helicobacter pylori, Helicobacter suis, Klebsiella species, Klebsiella pneumoniae, Lactobacillus acidophilis, Lawsonia intracellularis, Legionella species, Legionella pneumophilia, Leptospira species, such as Leptospira canicola, Leptospira grippotyposa, Leptospira hardjo, Leptospira borgpetersenii hardjo-bovis, Leptospira borgpetersenii hardjo-prajitno, Leptospira interrogans, Leptospira icterohaemorrhagiae, Leptospira urviv, Leptospira, Leptospira urviving, Listeria species, Listeria monocytogenes, Meningococcal bacteria, Moraxella species, Mycobacterium species, Mycobacterium bovis, Mycobacterium tuberculosis, Mycobacterium avium, Mycobacterium intracellular, Mycobacterium kansaii, Mycobacterium gordonae, Mycoplasma species, such as, Mycoplasma hyopneumoniae, Mycoplasma synoviae, Mycoplasma hyorhinis, Mycoplasma pneumoniae, Mycoplasma mycoides subsp. Mycoides LC, Neisseria species, Neisseria gonorrhoeae, Neisseria meningitidis, Odoribacter denticanis, Pasteurella species, Pasteurelta (Mannheimia) haemolytica, Pasteurella multocida, Photorhabdus luminescens, Porphyromonas gingivalis, Porphyromonas gulae, Porphyromonas salivosa, Propionibacterium acnes, Proteus species, Proteus vulgaris, Pseudomonas species, Pseudomnas wisconsinensis, Pseudomonas aeruginosa, Pseudomonas fluorescens C9, Pseudomonas fluorescens SIKW1, Pseudomonas tragi, Pseudomonas luteola, Pseudomonas oleovorans, Pseudomonas s B11˜1, Psychrobacter immobilis, Rickettsia spp, Rickettsia prowazekii, Rickettsia rickettsia, Salmonella species, Salmonella bongori, Salmonella choleraeuis, Salmonella urviv, Salmonella enterica, Salmonella urvivi, Salmonella typhimurium, Salmonella typhi, Serratia marcescens, Shigella species, Spirlina plaiensis, Staphylococci species, Staphlyoccocus aureus, Staphylococcus epidermidis, Staphylococcus hyicus, Streptococcus species, Streptobacillus moniliformis, beta-hemolytic Streptococcus, Streptococcus pyogenes (Group A Streptococcus), Streptococcus agalactiae (Group B Streptococcus), Streptococcus (viridans group), Streptococcus faecalis, Streptococcus bovis, Streptococcus uberis, Streptococcus dysgalactiae, Streptococcus (anaerobic sps.), Streptococcus pneumoniae, Streptococcus mutans, Streptococcus sobrinus, Streptococcus sanguis, Streptomyces albus, Streptomyces cinnamoneus, Streptomyces exfoliates, Streptomyces scabies, Sulfolobus acidocaldarius, Syechocystis sp., Treponena species, Treponema denticola, Treponema minutum, Treponema palladium, Treponema pertenue, Treponema phagedenis, Treponema refringens, Treponema vincentii, Vibrio species, Vibrio cholerae, and Yersinia species.

The antigenic proteins or peptides are very often surface proteins, lipo proteins or glycoproteins from the pathogenic bacteria, such as any of those mentioned above, in particular bacteria causing disease in mammals, for example surface proteins, lipo proteins or glycoproteins from Mycobacterium, Haemophilus, Mycoplasma, Streptococcus, and Glasserela species. Antigenic proteins or peptides of bacterial pathogens include, but are not limited to, an iron-regulated outer membrane protein (IROMP), an outer membrane protein (OMP), and an A-protein of Aeromonis salmonicida which causes furunculosis, p57 protein of Renibacterium salmoninarum which causes bacterial kidney disease (BKD), major surface associated antigen (msa), a surface expressed cytotoxin (mpr), a surface expressed hemolysin (tsh), and a flagellar antigen of Yersiniosis; an extracellular protein (ECP), an IROMP, and a structural protein of Pasteurellosis; an OMP and a flagellar protein of Vibrosis anguillarum and V. ordalir, a flagellar protein, an OMP protein, aroA, and purA of Edwardsiellosis urviving and E. tarda; and surface antigen of Ichthyophthirius; and a structural and regulatory protein of Cytophaga columnari; and a structural and regulatory protein of Rickettsia, IsdA, Cif A, C!fB, Opp3A, HLA and capsular polysaccharides from Staphylococcus aureus. Any of these polypeptides, their combinations or antigenic fragments may form part of the particle (granule or aggregate) of the invention.

Non-limiting viruses from which the antigenic proteins or peptides may originate include Avian herpesvirus, Avian influenza, Avian leukosis virus, Avian paramyxoviruses, Border disease virus, Bovine coronavirus, Bovine ephemeral fever virus, Bovine herpes viruses, Bovine immunodeficiency virus, Bovine leukemia virus, Bovine parainfluenza virus 3, Bovine respiratory syncytial virus, Bovine viral diarrhea virus (BVDV), BVDV Type I, BVDV Type II, Canine adenovirus, Canine coronavirus (CCV), Canine distemper virus, Canine herpes viruses, Equine herpes viruses, Canine influenza virus, Canine parainfluenza virus, Canine parvovirus, Canine respiratory coronavirus, Classical swine fever virus, Eastern Equine encephalitis virus (EEE), Equine infectious anemia virus, Equine influenza virus, West nile virus, Feline Calictvirus, Feline enteric coronavirus, Feline immunodeficiency virus, Feline infectious peritonitis virus, Feline herpes Virus, Feline influenza virus, Feline leukemia virus (FeLV), Feline viral rhinotracheitis virus, Lentivirus, Marek's disease virus, Newcastle Disease virus, Ovine herpesviruses, Ovine parainfluenza 3, Ovine progressive pneumonia virus, Ovine pulmonary adenocarcinoma virus, Pantropic CCV, African swine fever virus, Classical swine fever virus, Foot and Mouth disease virus, Porcine coronaviruses, Porcine circovirus (PCV) Type I, PCV Type II, Porcine epidemic diarrhea virus, Porcine hemaggiutinating encephalomyletitis virus, Porcine herpesviruses, Porcine parvovirus, Porcine reproductive and respiratory syndrome (PRRS) Virus, Pseudorabies virus, Rabies, Rotavirus, Rhinoviruses, Rinderpest virus, Swine influenza virus, Transmissible gastroenteritis virus, Turkey coronavirus, Venezuelan equine encephalitis virus, Vesicular stomatitis virus, West Nile virus, Western equine encephalitis virus, Adenoviridae (most adenoviruses); Arena viridae (hemorrhagic fever viruses); Asfavirus (African swine fever virus as its only member) Astroviruses; Bungaviridae (e.g., Hantaan viruses, bunga viruses, phleboviruses and Nairo viruses); Calciviridae (e.g., Rabbit haemorrhagic virus and other viral strains that cause gastroenteritis); Coronoviridae (e.g., coronaviruses); Porcine Diarrheal virus, Porcine gastroenteritis virus, Middle East respiratory syndrome coronavirus (MERS-CoV), Severe acute respiratory syndrome coronavirus 1 (SARS-CoV-1), Severe acute respiratory syndrome coronavirus 2 (SARS-CoV-2), Filoviridae (e.g., ebola viruses); Flaviridae (e.g., Classical swine fever virus, hepatitis C virus, dengue viruses, encephalitis viruses, yellow fever viruses); Hepadnaviridae (Hepatitis B virus); Herpesviridae (Porcine Pseudorabies virus (Aujeszky), herpes simplex virus (HSV) 1 and 2, varicella zoster virus, cytomegalovirus (ClvIV), herpes virus); Iridovtridae; Norwalk and related viruses; Orthomyxoviridae (e.g., influenza viruses); Papovaviridae (papilloma viruses, polyoma viruses); Paramyxoviridae (e.g., parainfluenza viruses, mumps virus, measles virus, respiratory syncytial virus); Parvovirida (parvoviruses); Picomaviridae (e.g., Foot and Mouth disease virus, polio viruses, hepatitis A virus; enteroviruses, human Coxsackie viruses, rhinoviruses, echovtruses); Poxviridae (variola viruses, vaccinia viruses, monkeypox virus and other pox viruses); Reovirtdae (e.g., reoviruses, orbiviurses and rotaviruses); Retroviridae (e.g. human immunodeficiency viruses, such as HIV-1or HIV-2 (also referred to as HTLV-III, LAV or HTLV-III/LAV, or HIV-III; and other isolates, such as HI-LP); Rhabdoviradae (e.g., vesicuiar stomatitis viruses, rabies viruses); Togaviridae (e.g., equine encephalitis viruses, rubella viruses); and Unclassified entities such as the etiological agents of Spongiform encephalopathies, the agent of delta hepatitis (thought to be a defective satellite of hepatitis B virus).

Non-limiting parasites from which the antigenic proteins or peptides may originate include Anaplasma, Ancylostoma (hookworms), Ascaris, Babesia, Coccidia, Cryptosporidium parvum, Dirofilaria (heartworms), Eimeria species, Fasciola hepatica (liver fluke), Giardia, Hammondia, Isopsora, Leishmania species, Neospora caninum, Plasmodium species, Sarcocystis, Schistosoma, Strongy ides, Taenia, Toxoplasma gondii, Trichineila species, Trichomonas species, Trypanosoma species, and external parasites such as ticks, for example Ixodes, Rhipicephalus, Dermacentor, Ambiyomma, Boophilus, Hyaiomma, and Haemaphysaiis species.

In the sense of this description, “self-antigens” (or autoantigens) are particles that the immune system recognizes as part of the body it protects. The ability of the immune system to differentiate between self- and non-self antigens is called, self/non-self discrimination in immunology. However, in certain circumstances mistakes in the recognition of benign environmental (self-antigens) as dangerous, elicit an immune response against anything in the body that expresses that antigen. This process is how autoimmune disease, allergies and some cancers develop.

In other embodiments invention, an antigen is an allergen. An “allergen” or “allergenic antigen” is a type of antigen that produces an abnormally vigorous immune response in which the immune system fights off a perceived threat that would otherwise be harmless to the body. Such reactions are called allergies. In 30 technical terms, an allergen is an antigen that is capable of stimulating a type-I hypersensitivity reaction in atopic individuals through immunoglobulin E (IgE) responses. Most humans mount significant Immunoglobulin E responses only as a defense against parasitic infections. However, some individuals may respond to many common environmental antigens. This hereditary predisposition is called atopy. In atopic individuals, non-parasitic antigens stimulate inappropriate IgE production, leading to type I hypersensitivity. Allergens can be found in a variety of sources, such as dust mite excretion, pollen, pet dander, even royal jelly, insect venoms, fungal spores and drugs (e.g. penicillin). Food allergies are not as common as food sensitivity, but some foods such as peanuts (a legume), nuts, seafood and shellfish are the cause of serious allergies in many people.

Non-limiting allergens that may be included in the particle of the invention are to proteins or peptides specific to the following genuses: Agropyron (e.g. Agropyron repens); Agrostis (e.g. Agrostis alba); Alder, Ainus (Ainus gultinoasa); Alternaria (Alternaria alte nata); Ambrosia (Ambrosia artemiisfolia; Anthoxanthum (e.g. Anthoxanthum odoratum); Apis (e.g. Apis multiflorum); Arrhenatherum (e.g. Arrhenatherum elatius); Artemisia (Artemisia vulgaris); Avena (e.g. Avena sativa); Betula (Betula verrucosa); Biattelia (e.g. Biattella germanica); Bromus (e.g. Bromus inermis); Canine (Canis familiaris); Chamaecyparis (e.g. Chamaecyparis urviv); Cryptomeria (Cryptomeria japonica); Cupressus (e.g. Cupressus sempervirens, Cupressus arizonica and Cupressus macrocarpa); Dactylis (e.g. Dactylis glomerata); Dermatophagoides (e.g. Dermatophagoides urviv); Felis (Felis domesticus); Festuca (e.g. Festuca elatiofy Holcus (e.g. Holcus lanatus); Juniperus (e.g. Juniperus sabinoides, Juniperus virginiana, Juniperus communis and Juniperus ashei); Lolium (e.g. Lolium perenne or Lolium multiflorum); Oiea (Olea urviv); Parietaria (e.g. Parietaia officinalis or Parietaria urvivi); Paspalum (e.g. Paspalum notatum); Periplaneta (e.g. Peripianeta americana); Phalaris (e.g. Phalaris arundinacea); Phieum (e.g. Phieum pratense); Plantago (e.g. Plantago lanceolata); Poa (e.g. Poa pratensis or Poa compressa); Quercus (Quercus alba); Secale (e.g. Secale cereale); Sorghum (e.g. Sorghum halepensis); Thuya (e.g. Thuya orientalis); and Triticum (e.g. Triticum aestivum), and combinations thereof.

As the skilled person in the art will understand the classification of the antigens includes overlapping zones, such as for example a venom toxin can be also classified as an allergen.

A “cancer antigen” or “tumor antigen” is an antigenic substance produced in tumor cells, i.e., it triggers an immune response in the host. There are different kinds of cancers that express different tumoral antigens. They can be self-antigens that either mutate or recombine, creating antigens that are uniquely expressed in cancer cells, becoming useful tumor markers in identifying tumor cells with diagnostic tests and are potential candidates for use in cancer therapy. Alternatively, cancer might be provoked by self-antigens that without any change in their amino acid composition become overexpressed or expressed in abnormal locations, causing the uncontrolled growth of the cells. Despite the risks implied, the benefits afforded compensate the development of novel immunotherapy strategies and vaccines target against this kind of self-antigens. The field of cancer immunology studies such topics.

In some embodiments of the invention, the antigenic proteins or peptides is a tumor antigen. In some embodiments, the tumor antigen selected from one or more of p53, BCR-ABL, glycoprotein 100 (gp100), mucine-1 (MUC-1), carcinoembryonic antigen (CEA), guanylyl cyclase C, NY-ESO-1, human telomerase reverse transcriptase (hTERT), alpha lactalbumin, prostate specific membrane antigen (PMSA), WNT1, CTLA-4, programme death 1 antigen and ligand (PD-1, PD-L1), KRAS mutated, WT1, MUC1, LMP2, HPV E6 or HPV E7, EGFR or variant form, for example, EGFRvIII, HER-2/neu, Idiotype, MAGE A3, p53 nonmutant, 35 NY-ESO-1, PSvlA, GD2, CEA, MelanA/MART1, Ras mutant, gp100, p53 mutant, Proteinase3 (PR1), bcr-ab1, Tyrosinase, Survivin, PSA, hTERT, Sarcoma translocation breakpoints, EphA2, PAP, ML-IAP, AFP, EpCAM, ERG (TMPRSS2 ETS fusion gene), NA17, PAX3, ALK, Androgen receptor, Cyclin B1, polysiailic acid, MYCN, RhoC, TRP-2, GD3, Fucosyl GM1, Mesothelin, PSCA, MAGE A1, sLe (animal), CYP1 B1, PLAC1, GM3, BORIS, Tn, GloboH, ETV6-AML, NY-BR-1, RGS5, SART3, STn, Carbonic anhydrase IX, PAX5, OY-TES1, Sperm protein 17, LCK, HMWMAA, AKAP-4, SSX2, XAGE 1, B7H3, Legumain, Tie 2, Page4, VEGFR2, MAD-CT-1, FAP, PDGFR-beta, MAD-CT-2, or Fos-related antigen 1. In some embodiments, the tumor antigen is one or more of urviving, Her-2, EFGRvIiI, PSA, PAP or PMSA.

In some embodiments of the invention, an antigen is a self antigen. In some embodiments, a self antigen is an antigen of a subject's own cells or cell products that causes an immune response in a subject. In some embodiments, a self antigen includes, but is not limited to, a tumor antigen, an antigen associated with Alzheimer's Disease, an antigen against an antibody, or an antigen that is expressed from human endogenous retroviral elements. An antigen associated with Alzheimer's Disease may be tau or p-amyloid. An antigen against an antibody may be an antigen against a human antibody, for example, in some embodiments the antigen is IgE.

In another particular embodiment, when cancer antigens of the previous list are the proteins and/or peptide molecules in the immunogenic composition, they form part of fusion proteins comprising one or more of them fused to fragments that target a particular antigen presenting cell. The skilled person in the art knows the common techniques of molecular biology for the construction and obtention of the said fusion proteins.

The particle comprised in the immunogenic composition or consisting of it, is a nanoparticle or a microparticle. The term “nanoparticle” as used herein, refers to a particle with at least two dimensions at the nanoscale, particularly with all three dimensions at the nanoscale. For analogy, the term “microparticle” as used herein, refers to a particle with at least two dimensions at the microscale, particularly with all three dimensions at the microscale. In a particular embodiment, the particle is from 50 nm to 4000 nm (i.e., 4.0 micrometers), in particular from 200 nm to 4000 nm (i.e., 1.5 micrometers). In a particular embodiment the size of the particle is from 200 nm to 2000 nm, or from 700 to 1000, and in particular selected from 700, 750, 800, 850, 900, 950 and 1000 nm, preferably measured as the hydrodynamic diameter using Transmission Electron Microscopy (TEM) (see below for details).

On the other hand, the nanoparticles released under the above indicated physiological conditions are, in a more particular embodiment, nanoparticles of also assembled proteins/peptides with a hydrodynamic diameter from 5 to 80 nm. More in particular from 5 to 50 nm. They have, in a more particular embodiment a size selected from 5, 6, 7, 8, 9, 10, 11, 12, 13, 14 and 15 nm. All sizes measured as the hydrodynamic diameter, preferably using Transmission Electron Microscopy (TEM) (see below for details).

As regards the shape of the nanoparticles or microparticles described herein, there are included spheres, polyhedral and rod-shape. Particularly, when the nanoparticle or microparticle is substantially rod-shaped with a substantially circular cross-section, such as a nanowire or a nanotube, microwire or microtube, the “nanoparticle” or “microparticle” refers to a particle with at least two dimensions at the nanoscale or microscale, these two dimensions being the cross-section of the nanoparticle or the microparticle.

In a particular embodiment of the first aspect, optionally in combination with any of the embodiments provided above or below, the particle is spherical or pseudospherical.

As used herein, the term “size” refers to a characteristic physical dimension. For example, in the case of a nanoparticle/microparticle that is substantially spherical, the size of the nanoparticle/microparticle corresponds to the diameter of the nanoparticle/microparticle. When referring to a set of nanoparticles/microparticles as being of a particular size, it is contemplated that the set can have a distribution of sizes around the specified size. Thus, as used herein, a size of a set of nanoparticles/microparticles can refer to a mode of a distribution of sizes, such as a peak size of the distribution of sizes. In addition, when not perfectly spherical (pseudospherical), the diameter is the equivalent diameter of the spherical body including the object. This diameter is generally referred as the “hydrodynamic diameter”. Said hydrodynamic diameter can be determined by methods well known to the skilled person, including Dynamic Light Scattering (DLS), for example using a Wyatt Möbius coupled with an Atlas cell pressurization system, or a a Zetasizer Advance Pro instrument (Malvern Instruments). In particular embodiments, the hydrodynamic diameter can be determined by Transmission Electron Microscopy (TEM) images, more in particular by field scanning electron microscopy (FESEM) obtained, for example, with a FESEM Zeiss Merlin. The size of the microparticle can be determined from the scale bar provided by the microscope using an imaging software, for example, the ImageJ software.

The particles of the immunogenic composition as previously disclosed are, indeed,
mechanically stable, which means that the self-assembled proteins/peptides with the divalent cations remain structured, which means that do not lose the tridimensionality configuration and maintain their hydrodynamic size when submitted at sonication conditions, for example including 5 rounds of 40 seconds; 0.5 of pulse on; 0.5 of pulse off and a wave width of 10% in a high intensity sonicator, with 3 mm-diameter titanium probe;
are in the form of a precipitated pellet in aqueous media, when centrifuged at 15.000 g at a temperature from 4° C. to 30 C; and
they have a release profile under physiologic conditions in phosphate buffered saline, in which an amount from 30% to 100% by weight in relation to the total weight of the proteins and/or peptide molecules is released in form of nanoparticles with a hydrodynamic diameter from 5 to 80 nm with a time from 24 h to 10 days.

For the test of the “mechanically stable” feature, the skilled person will know which sonicator use and to adapt to the rounds, time and pulses. In an example, the sonicator is a Branson sonifier 450 with 3 mm-diameter titanium probe, widely used in the laboratory, and the same is adjusted to output sonication conditions including 5 rounds of 40 seconds; 0.5 of pulse on; 0.5 of pulse off and a wave width of 10% in a high intensity.

As would be evident for the skilled reader, all embodiments disclosed above related to the particle in the immunogenic composition of the first aspect also apply to the particle of the fourth aspect.

A second aspect of the invention is a vaccine composition comprising a therapeutically effective amount (i.e., immunologically effective amount) of the immunogenic composition as defined in the first aspect, together with a pharmaceutically and acceptable excipient and/or carrier. All embodiments disclosed above for immunogenic composition of the first aspect also apply to this second aspect.

In a particular embodiment the vaccine further comprises and adjuvant. In another more particular embodiment, the adjuvant is selected the group consisting of alum, aluminium hydroxide, aluminium phosphate, Freund's complete adjuvant, squalene, monophosphoryl lipid A, the two-component liposomal adjuvant system CAF01, and combinations thereof.

CAF01 is a novel two-component liposomal adjuvant system composed of a cationic liposome vehicle (dimethyldioctadecyl-ammonium (DDA)) stabilized with a glycolipid immunomodulator (trehalose 6,6-dibehenate (TDB)) which is a synthetic variant of cord factor located in the mycobacterial cell wall.

In another particular embodiment of the vaccine of the invention, it is for administration selected from intramuscular administration, intradermal administration, subcutaneous administration, oral administration or aerial (i.e., nasopharyngeal) administration.

Suitable carriers, excipients, etc. for preparing the vaccines according to the invention can be found in standard pharmaceutical texts, and include, as a way of example preservatives, agglutinants, humectants, emollients, and antioxidants.

In a particular embodiment, optionally in combination with any embodiment above or below, the pharmaceutically acceptable excipient comprises any pharmaceutically acceptable component other than the immunogenic component. The carrier can be organic, inorganic, or both. Suitable carriers well known to those of skill in the art and include, without limitation, large, slowly metabolized macromolecules such as proteins, polysaccharides, polylactic acids, polyglycolic acids, polymeric amino acids, amino acid copolymers, lipid aggregates (such as oil droplets or liposomes) and inactive virus particles.

Additionally, if desired, the carrier can contain pharmaceutically acceptable auxiliary substances such as, for example, wetting agents, dispersing agents, emulsifying agents, buffering agents (for example, phosphate buffer), stabilizing agents such as carbohydrates (for example, glucose, sucrose, mannitol, sorbitol, starch, or dextran), or proteins (for example, albumin, casein, bovine serum, or skimmed milk).

The election of the formulation for the immunogenic composition, or pharmaceutical composition, or vaccine, may greatly depend upon the route of administration. Any route of administration may be used. In some embodiments, the route of administration is parenteral, for example, by intramuscular, intravenous, intraarterial, intraperitoneal, subcutaneous, or transdermal injection, and the composition is then appropriate for parenteral administration. Interestingly, the higher stability of the particles of the invention compared to soluble proteins allows for an optimal behavior in the aggressive mucosal environments. Therefore, in particular embodiments the route of administration is mucosal, for example oral or nasal/intranasal. This is advantageous, for instance, for the prevention of infections of the higher and lower respiratory tract. Topical administration is also contemplated, such that the composition may be a topical composition.

The compositions may be in any form, including, among others, tablets, pellets, capsules, aqueous or oily solutions, suspensions, emulsions, aerosols, or dry powdered forms suitable for reconstitution with water or other suitable liquid medium before use. For example, for parenteral or systemic administration the compositions may be in the form of an inyectable composition. For mucosal administration, such as intranasal administration, the composition may be in the form of an aerosol, a nebulizer, or a spray.

The compositions of the present invention for the proposed according to first and second aspects, can be prepared according to methods well known in the state of the art. The appropriate excipients and/or carriers, and their amounts, can readily be determined by those skilled in the art according to the type of composition being prepared.

Excipients usually used in vaccines include without any limitation any and all solvents, dispersion media, wetting agents, emulsifying agents, coatings, adjuvants, stabilizing agents such as carbohydrates (for example glucose, sucrose, mannitol, sorbitol, starch or dextran), diluents, buffer agents (for example phosphate buffer), proteins (for example albumin, casein, bovine serum or skimmed milk), preservatives, isotonic agents, adsorption delaying agents, and the like. In preferred embodiments, especially those that include lyophilized immunogenic compositions, stabilizing agents for use in the present invention include stabilizers for lyophilization or freeze-drying.

In a third aspect, the invention relates to the immunogenic composition as defined above, or the vaccine as defined above or the particle as described above for use as a medicament. All embodiments disclosed above for the immunogenic composition, vaccine or particle also apply to the third aspect.

In a particular embodiment the immunogenic composition or the vaccine for use as defined above are for use in the prevention and/or treatment of diseases caused by a pathogen, in particular selected from a virus, a bacterium, a parasite, a fungus, a protozoa and combinations thereof.

This can also be formulated as the use of the immunogenic composition or the vaccine as defined above for the manufacture of a medicament for the treatment or prevention of diseases caused by a pathogen, in particular selected from a virus, a bacterium, a parasite, a fungus, a protozoa and combinations thereof. The present invention also relates to a method for the treatment or prevention of diseases caused by a pathogen, in particular selected from a virus, a bacterium, a parasite, a fungus, a protozoa and combinations thereof, comprising administering a therapeutically pharmaceutically effective amount of the immunogenic composition or the vaccine as defined above, together with pharmaceutically acceptable excipients or carriers, in a subject in need thereof, including a human.

Also disclosed herein is a method of preparing the immunogenic composition of the first aspect or the particle of the fourth aspect, said method comprising the following steps:
(a) mixing in a recipient one or more types of proteins and/or peptides in a polar solvent;
(b) adding one or more salts of divalent cations to the proteins and/or peptides at a final a final ratio of moles of salt of divalent cation:moles of protein comprised from 40:1 to 1000:1 while agitating the mixture;
(C) allowing to form a precipitate and colloidal suspension comprising assembled protein and/or peptide molecules with the divalent cations;
(d) isolating the assembled protein and/or peptides with the divalent cations, and
(e) optionally resuspending them in a fresh buffered solvent.

Also disclosed is a particle obtainable by steps (a)-(d) of a method as defined above. Also disclosed is an immunogenic composition obtainable by steps (a)-(e) of a method as defined above. All embodiments disclosed above for the immunogenic composition of the first aspect, or for the particle of the fourth aspect also apply to their preparation method.

In a particular embodiment, step (c) of the method is carried out by means selected from the group consisting of centrifugation, filtering, drying, and combinations thereof, the skilled person will know. In another more particular embodiment isolation is performed by means of centrifugation.

In a particular embodiment of the methods, the polar solvent of step (a) is an aqueous buffered composition, more in particular an aqueous buffered composition at a pH from 6.8 to 7.5. More in particular is water with a buffer to adjust pH. Particular buffers include phosphate-buffered saline (containing disodium hydrogen phosphate, sodium chloride and, in some formulations, potassium chloride and potassium dihydrogen phosphate), Tris-glycine or Tris-HCl. Aqueous buffered compositions area also termed in this description as an “aqueous media”.

Indeed, is also herewith disclosed an immunogenic composition comprising or consisting of a particle with a hydrodynamic diameter from 200 nm to 4000 nm, said particle comprising antigenic protein and/or peptides and one or more salts of divalent cations, and wherein the antigenic protein and/or peptides remain assembled (i.e., self-assembled) in the presence of the said salts of divalent cations, being the ratio of moles of salt of divalent cation:moles of protein and/or peptide molecules in the particle comprised from 40:1 to 1000:1; wherein said immunogenic composition is obtainable by the above indicated methodological steps.

Other more particular modes of preparing the particles comprised or consisting in the immunogenic composition of the invention are disclosed in the previously referenced documents, the international patent application with the publication number WO2020208065A1, and the document of Lopez-Laguna et al., “In Vitro Fabrication of Microscale Secretory Granules”, Advanced Functional Materials Journal-2021, 210914, https://doi.org/10.1002/adfm.202100914.

In another particular embodiment of the method of preparing the particle, immunogenic composition or of the particle or immunogenic composition obtainable by the method, the final ratio of moles of salt of divalent cation:moles of protein is comprised from 40:1 to 800:1. More in particular from 40:1 to 500:1, even more in particular is from 40:1 to 200:1. Particular preferred ratios are selected from 40:1, 50:1, 60:1, 70:1, 100:1 and 150:1. The skilled person will understand that these ratios, as well as those of 40:1 to 1000:1 will be maintained in the obtained nano- or microparticle defined according to the first aspect.

Particular salts of divalent cations are the same as indicated for the first aspect of the invention. The skilled person in the art will also know that the mole ratios will depend on the protein that is going to be used, and the skilled man will know how to accommodate the amounts. For instance, the number of histidine residues or of other amino acid residues that can made complex linkages with the divalent cations will be a variable when adjusting the proportions, mainly requiring low amounts of divalent cations when the number of this complexing to the cation residues is higher in the protein molecule. Thus, it will vary depending on the amino acids in the protein molecule that can chelate (coordinated linkage) with the divalent cations. In a particular embodiment of the method of the second aspect when salts of divalent cations are used, the one or more proteins comprise one or more histidine residues.

The invention also proposes as an aspect a method for the preparation of a vaccine as defined in the second aspect, comprising the step of mixing the immunogenic composition of the first aspect or the particle of the fourth aspect with a pharmaceutically acceptable excipient and/or carrier. All embodiments disclosed above for the immunogenic composition, vaccine or particle also apply to this aspect.

As indicated, another aspect of the invention is a vaccination kit comprising:
(a) an immunogenic composition as defined in the first aspect or a particle as defined in the fourth aspect;
(b) a pharmaceutically acceptable excipient and/or carrier;
(c) optionally an adjuvant; and
(d) optionally instructions for its use.

All embodiments disclosed above for the immunogenic composition or particle also apply to this aspect.

As mentioned above, the immunogenic composition or the vaccine comprising it are capable of inducing a response by the immune system of the subject, which can further be used to prevent or treat diseases. Thus, a third aspect of the invention relates to an immunogenic composition as defined in the first aspect, or a vaccine as defined in the second aspect, or a particle as defined in the fourth aspect, or a vaccination kit as defined above, for use as a medicament (i.e., for use in therapy). In particular embodiments the immunogenic composition, particle, vaccine or vaccination kit is for use in the prevention and/or treatment of diseases caused by a pathogen, in particular selected from a virus, a bacterium, a fungus, a protozoa and combinations thereof. As used herein, the term “treating” includes a reduction or prevention of the development or progression of the disease or disorder or symptoms, as well as the reduction or elimination of an existing disease or disorder or symptoms. The “treatment” can also include a prophylactic treatment. In a particular embodiment, the immunogenic composition, particle, vaccine, or vaccination kit is for use in the prevention and/or treatment of infections. In a more particular embodiment, the immunogenic composition, particle, vaccine, or vaccination kit is for use in the prevention of infections. In more particular embodiment, the disease or infection is caused by a virus, for example, by the African swine fever virus.

In other embodiments, the immunogenic composition, particle, vaccine, or vaccination kit is for use in the prevention and/or treatment of cancer. In other embodiments, the immunogenic composition, particle, vaccine or vaccination kit is for use in the prevention and/or treatment of allergies. The antigenic proteins or peptides forming the particle will determine the disease to be treated or prevented. For example, for treating a disease or infection caused by the African swine fever virus, the antigenic protein may be p32.

Throughout the description and claims the word “comprise” and variations of the word, are not intended to exclude other technical features, additives, components, or steps. Furthermore, the word “comprise” encompasses the case of “consisting of”. Additional objects, advantages and features of the invention will become apparent to those skilled in the art upon examination of the description or may be learned by practice of the invention. The following examples are provided by way of illustration, and they are not intended to be limiting of the present invention. Furthermore, the present invention covers all possible combinations of particular and preferred embodiments described herein.

### EXAMPLES

Example 1. Experimental immunization of pigs with “protein only secretion granules with vaccine purposes (POMVs from now on)” induce humoral and specific T-cell responses. In vivo assay with swine and a vaccine with the p32 protein of the African swine fever virus.

The main goal of this experiment was to provide enough experimental evidence of the efficacy of POMV (i.e., protein and/or peptide molecules and one or more salts of divalent cations

Previous results from the inventors confirmed that immunization of pigs with monomeric soluble p32, one of the most antigenic proteins from African swine fever virus (ASFV encodes more than 150 proteins), failed at inducing specific antibody responses unless it was combined with exogenous adjuvants. Independently of the adjuvant used, no specific CD8 T-cells were detected.

With this data at hand, it was decided to immunize pigs with the novel p32 granule formulation prepared as described.

RK-Linker-p32-H6 protein of SEQ ID NO: 1 (that is, with a 4 RK cationic domain at the amino terminus) was initially adjusted to 1 mg/mL and subsequently aliquoted into fixed final volumes of 500 μL. Next, a 0.22 μM-filtered solution of ZnCl2 (400 mM stock) was added to each Eppendorf tube, yielding a precisely defined final concentration of 10 mM. The resulting mixtures were then gently mixed, incubated for 10 min at room temperature, and subjected to centrifugation for 5 minutes at 10,000 g to isolate the soluble and insoluble fractions for further analysis. The remaining protein in the soluble fraction was quantified by means of the Bradford assay, thereby allowing for the accurate estimation of the percentage of precipitated protein. Finally, the obtained protein pellets (containing the secretory granules) were carefully stored at −80° C. for further use. At the moment to be used the pellets are resuspended in a volume of 200-300 μL. The resuspension buffer depends on the protein: for RK-Linker-p32-H6 166 mM NaCO3H+333 mM NaCl. For GFP-H6 166 mM NaCO3H. GFP-H6 of granules (SEQ ID NO: 2) were prepared with the same protocol of assembly as RK-Linker-p32-H6.

(SEQ ID NO: 1)
MRKRKRKRKGGSSRSSDFILNISMKMEVIFKTDLRSSSQWVFHAGSLYNW
FSVEIINSGRIVTTAIKTLLSTVKYDIVKSARIYAGQGYTEHQAQEEWNM
ILHVLFEEETESSASSENIHEKNDNETNECTSSFETLFEQEPSSEVPKDS
KLYMLAQKTVQHIEQYGKAPDFNKVIRAHNFIQTIYGTPLKEEEKEWVRL
MVIKLLKKISFYLTYIKHHHHHH.
(SEQ ID NO: 2)
MSKGEELFTGWPILVELDGDVNGHKFSVSGEGEGDATYGKLTLKFICTTG
KLPVPWPTLVTTLTYGVQCFSRYPDHMKRHDFFKSAMPEGYVQERTISFK
DDGNYKTRAEVKFEGDTLVNRIELKGIDFKEDGNILGHKLEYNYNSHNVY
ITADKQKNGIKANFKIRHNIEDGSVQLADHYQQNTPIGDGPVLLPDNHYL
STQSALSKDPNEKRDHMVLLEFVTAAGITHGMDELYKHHHHHH.

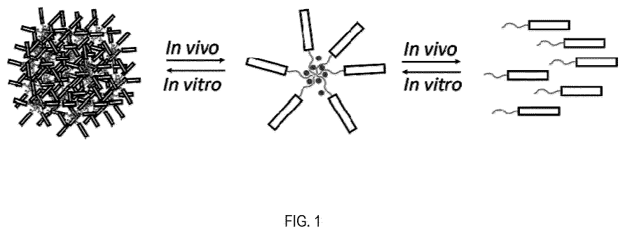

FIG. 1 illustrates in a schematic view, the particles or secretory granules (POMVs), which secrete particles of lower size that in turn ultimately disintegrate in the monomeric or oligomeric integrating proteins of the particle constituting the immunogen.

The obtained secretion granules were characterised as follows.

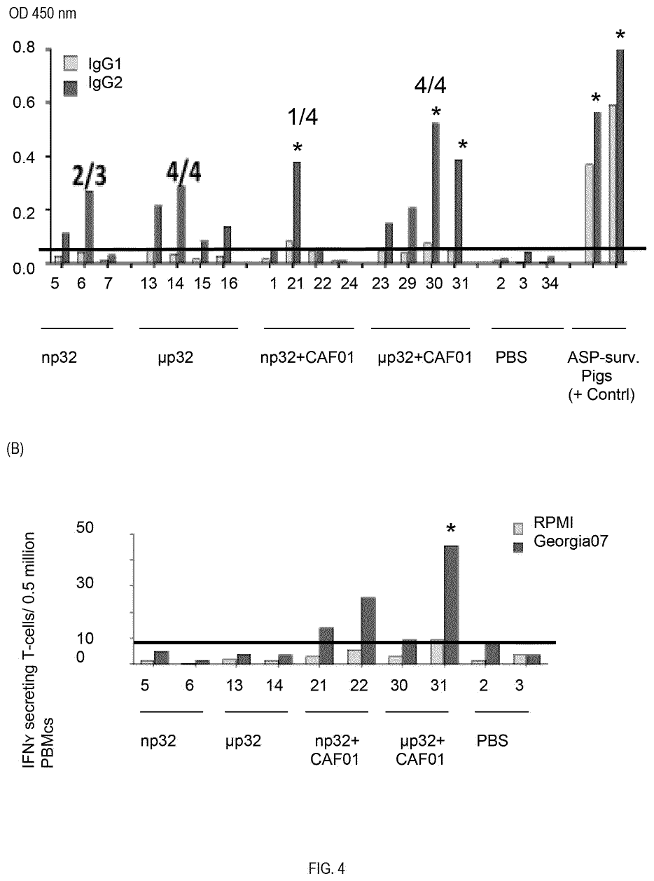

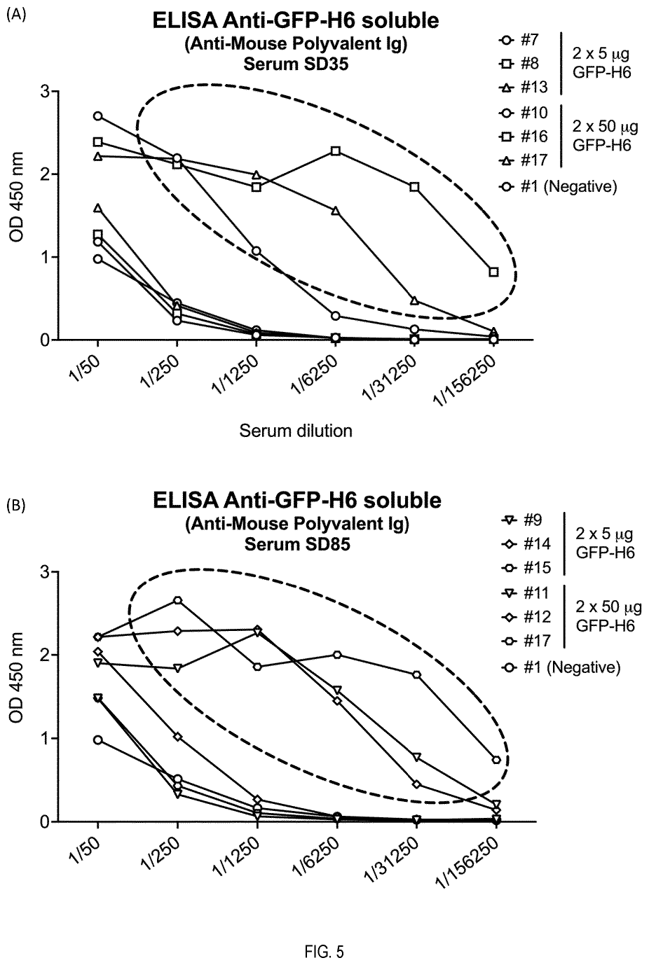

The hydrodynamic size of the secretion granules was determined by two methods: 1—dynamic light scattering (DLS; at 25° C. and 633 nm wavelength) in a Zetasizer Advance Pro instrument (Malvern Instruments) and expressed as mean±standard error (results shown in FIG. 7), and 2—field scanning electron microscopy (FESEM; at 10.00 to 25.00 K×magnifications and operating at 2.00 kV) in a FESEM Zeiss Merlin (Zeiss) without coating and using a high resolution secondary detector (results shown in FIG. 8). The size of the microparticle was determined from the scale bar provided by the microscope using the ImageJ software. According to both methods, the hydrodynamic size of the secretion granules is around 713.2±126.8 nm for RK-Linker-p32-H6 and around 712.3±276.4 nm for GFP-H6.

The mechanical stability was determined by sonication (5 rounds of 40 sec; 0.5 pulse on and off, wave width of 10%, Branson sonifier 450, 3 mm-diameter titanium probe). It was found that both RK-Linker-p32-H6 and GFP-H6 secretion granules maintained their hydrodynamic size after the sonication treatment, and thus were mechanically stable.

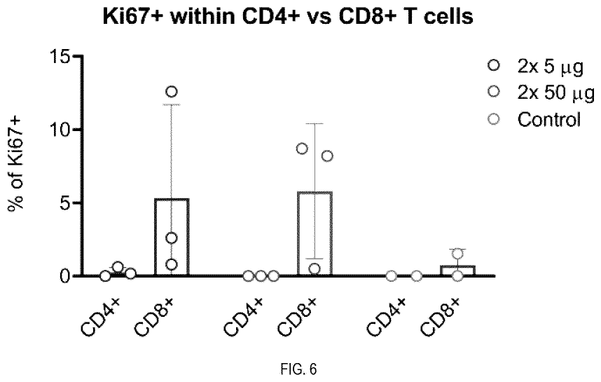

Thermal stability was studied by determining hydrodynamic size by DLS (as above) of the secretion granules after increasing temperatures from 4 to 50° C. (for 10 min at each temperature). Results in FIG. 9 show that it was found that both RK-Linker-p32-H6 and GFP-H6 secretion granules maintained their hydrodynamic size after after thermal treatment.

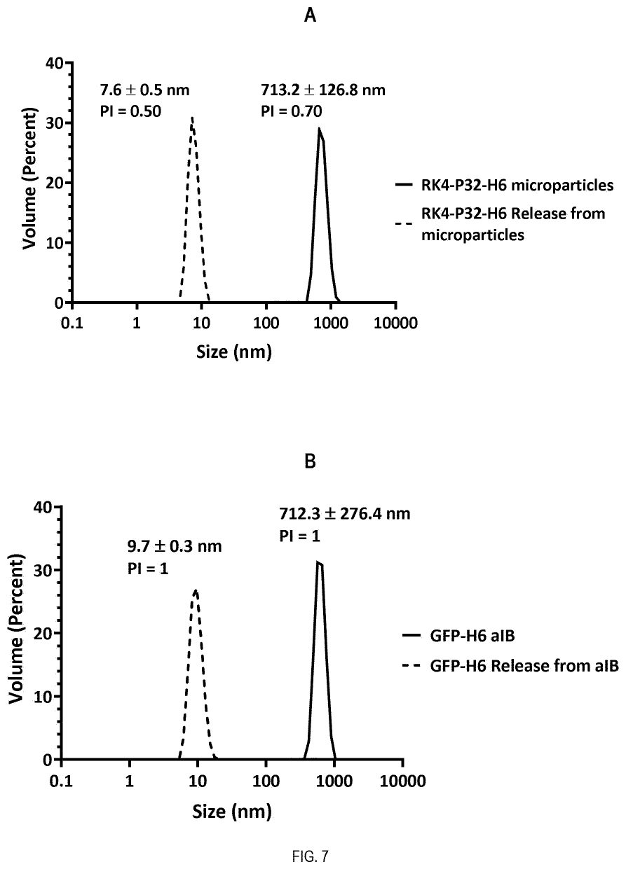

When centrifuged at 10,000 for 5 min at room temperature the granules are in the form of a dried precipitated pellet (FIG. 10).

The release profile of the the RK-Linker-p32-H6 and GFP-H6 secretion granules was studied at 0, 1, 3 and 7 days following the subsequent protocol. The obtained RK-Linker-p32-H6 and GFP-H6 secretion granules were thawed and gently resuspended in 500 μL of phosphate w/out salt solution at room temperature.

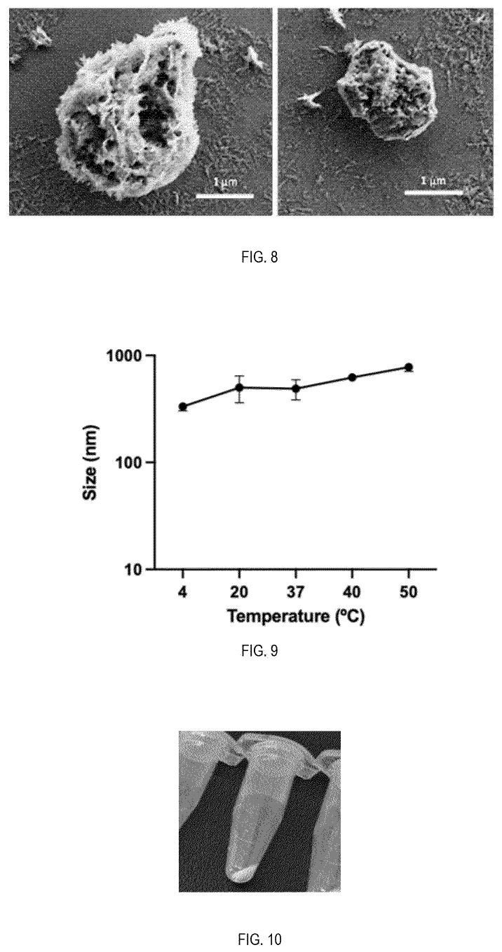

Granule suspensions were then incubated at 37° C. without agitation, and 80 μL aliquots were collected at several time points (0, 1, 3 and 7 days). Following centrifugation at 15,000 g for 15 min, soluble and insoluble fractions were separated, and the presence and integrity of proteins were determined using SDS-PAGE using TGX Stain-Free FastCast Acrylamide Kit, 12% (BioRad). Protein bands were additionally immunodetected by western blot (WB) and their intrinsic intensity quantified with Image Lab software, allowing the calculation of the percentage of released protein. Results are shown in FIG. 11. The released particles had a size of around 7.6±0.5 nm for RK-Linker-p32-H6 and of 9.7±0.3 nm for GFP-H6. In addition, no protein degradation was observed after seven days by SDS-PAGE, confirming that the protein was not degraded.

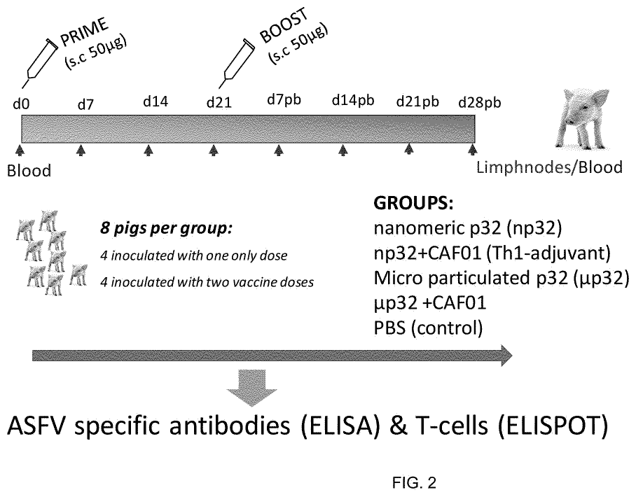

In FIG. 2 a schematic representation of the in vivo immunization procedure is provided.

As described groups of eight pigs each, were subcutaneously immunized with 50 micrograms (pg) of the corresponding protein in the absence or in the presence of CAF01 (following the instructions of the provider), adjuvant capable to induce Th1-like and CTL responses, an immune response crucial for ASF protection. Half of the pigs from each group (four) were immunized with only one dose and the other half were boosted with a second dose of the vaccine. Blood was taken weekly, and the sera was used in an ELISA to detect the level of specific antibodies against ASFV and their PBMCs were used to analyze the induction of IFNγ in response to specific in vitro stimulation with ASFV by ELISPOT. 21 days after the boost all pigs were euthanized and the most adjacent lymph nodes to the site of the injection were collected for further ELISPOT analysis.

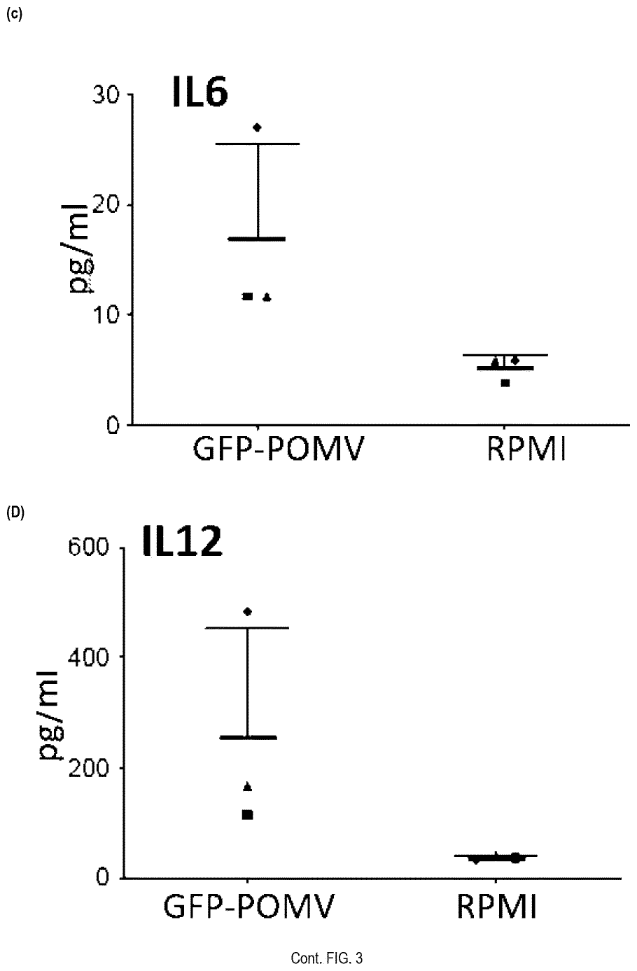

In contrast to that described previously for soluble p32, nanoparticulated p32 (np32, 90 nm hydrodynamic diameter) induced detectable ASFV-specific IgG responses immunized pigs, detectable from day 7 after the boost (pb), peaking at day 14pb to slowly decline by day 28pb. Interestingly, two of the three immunized pigs induced detectable anti-ASFV IgG2 immunoglobulins and no detectable IgG1 (FIG. 4 (A)), showing a clear Th1-like bias. The addition of the CAF01 adjuvant did not increase the percentage of responding pigs (¼) but seemed to increase the number of antibodies present in the responding animal (*). Interestingly, pigs immunized with the POMVs (i.e., particles of self-assembled protein, also termed herewith “μp”, prepared and characterised as described above) containing μp32 alone (three out of four) or in the presence of the CAF01 (all four), responded, the latter showing higher averages of ASFV-specific IgG2 (FIG. 4(A*)). The detection of specific antibodies in μp32-immunized pigs became evident at day 14pb (also the peak of the response), one week later than in np32-immunized pigs, most probably reflecting the slow delivery of np32 from POMVs as described before for other therapeutic proteins. The total bias of the immune response towards a Th1-like response (characterized by the isotype of immunoglobulins induced), contrasts with the mixed IgG1/IgG2 profile typically observed after vaccination with an ASFV live attenuated vaccine prototype available in the laboratory (right columns in figure A). Sera from LAV vaccinated pigs serve as control of the techniques but are not that useful for comparative responses since the antibodies detected in this case are directed against many proteins contained in the antigen-coated ELISA plates and not only against p32.

Pigs immunized with either np32 or μp32 in the presence of CAF01 induced T-cells that specifically secreted IFN-γ in response to overnight stimulation with ASFV, detectable in lymph nodes at the time of sacrifice (day 28 pb), confirming for the first time, the induction of Th1-like responses after immunization with p32 (FIG. 4 (B)). Lack of detectable responses in the absence of CAF01 was likely due to a limitation of the detection method.

In conclusion, two immunizations with 50 μg of μp32 in the presence of CAF01, demonstrated to induce the best ASFV-specific Th1-like responses of the formulations tested. The small amount of protein used in this proof of concept (POC) test (50pg), compared with the 1 mg dose used in mice for gene-therapy purposes open the door for optimal vaccine formulations just by increasing the vaccine dose. Future experiments can be thought to determine if vaccination with optimal concentrations of μp32 would need boosting and/or the presence of CAF01 or any other adjuvant, but these are in any case optimizations or variants under the rationale of the invention.

According to this example, it can be concluded that the immunogen of the invention, also herewith named POMVs or pp induces better Th-1like responses than its soluble nano-particulated counterpart. The responses were improved when the adjuvant CAF01 was used in the vaccine.

Example 2. Secretory granules stimulate the immune system in non-specific manner. Experiment performed. In-vitro with PBMCs from pigs.

Aiming to understand the mechanisms explaining the efficacy of the protein only microparticles (i.e., self-assembled proteins with the salts of divalent cations) in vivo, a experiment was performed, using Peripheral Blood Mononuclear Cells (PBMCs) from pigs immunized with BA71ACD2, a protective recombinant live attenuated vaccine available in the laboratory (Monteagudo P L, Lacasta A, Lopez E, Bosch L, Collado J, Pina-Pedrero S, Correa-Fiz F, Accensi F, Navas M J, Vidal E, Bustos M J, Rodriguez J M, Gallei A, Nikolin V, Salas M L, Rodriguez F. BA71ACD2: a New Recombinant Live Attenuated African Swine Fever Virus with Cross-Protective Capabilities. J Virol. 2017 Oct. 13; 91(21):e01058-17), as effector cells in an IFNγ-ELISPOT in vitro assay. Briefly, PBMCs from two pigs immunized with an optimal dose of BA71ACD2, were collected four weeks after immunization (memory phase) and they were stimulated in vitro overnight with either: RPMI (negative control), the non-specific stimulator Phytohemagglutinin (PHA), African swine fever virus (ASFV) as specific stimulus, p32 microparticles prepared and characterised as described above (p32-pp) or GFP microparticles prepared and characterised as described above (GFP-pp). Table 1 summarizes the results obtained.

TABLE 1
Number of IFNγ-positive cells detected after in vitro stimulation

| | RPMI | PHA | ASFV | p32-μp | GFP-μp |
|---|---|---|---|---|---|
| Pig #1 | 0 | +++++ | 150 | ++++ | ++++ |
| Pig #2 | 0 | +++++ | 240 | ++++ | ++++ |

As expected, RPMI did not stimulate the induction of IFNγ, while PHA stimulated very strong responses (uncountable spots, represented as ++++ responses), corresponding with the non-specific stimulation of different PBMC subsets. Also as expected, between 150-300 IFNγ positive spots (indicated as ++ stimulation) was observed after the in vitro stimulation with ASFV, corresponding with the detection of ASFV-specific memory T-cells presented in successfully vaccinated pigs. Interestingly, both p32-pp and GFP-pp stimulated the induction of IFNγ with identical efficiency, independently of the presence of the ASFV specific p32 protein or not. This result together with the fact that the number of specific cells were similar to those obtained with PHA confirm the non-specific nature of the stimulation. The non-specific nature of the responses induced by p32-pp and GFP-pp has been confirmed by using PBMCs from non-vaccinated naïve pigs. As expected, soluble GFP did not stimulate the induction of IFNγ demonstrating that the stimulation depends on its microparticulated nature of the immunogenic composition.

Example 3. Secretory granules stimulate the immune system in non-specific manner. Experiment performed In-vitro Pig Alveolar Macrophages (PAMs)

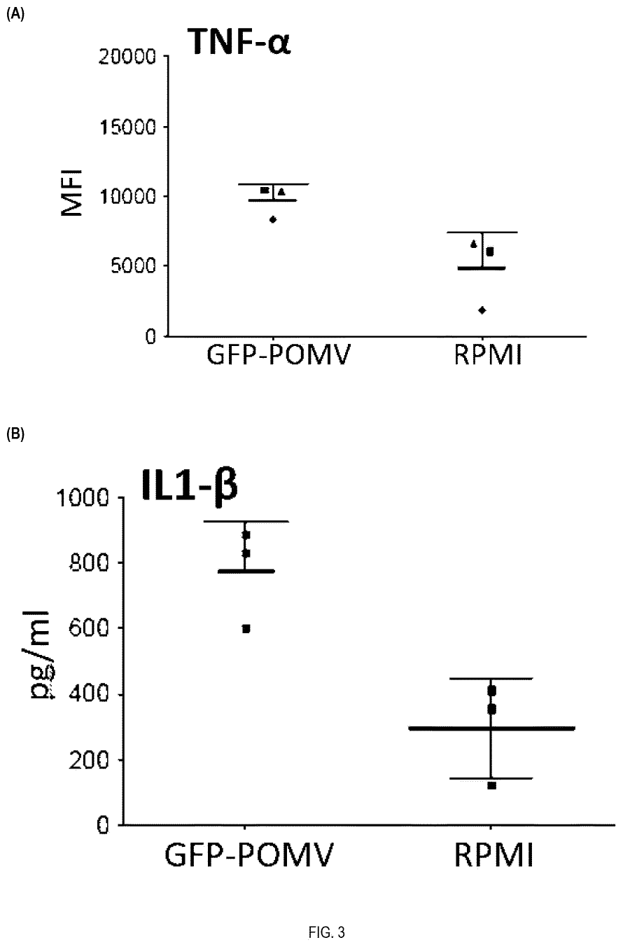

To confirm the potential of POMVs as non-specific immunostimulators, Lung (Alveolar) pig isolated macrophages (PAMs) were stimulated with the immunogen of the invention (prepared as in example Example 1), called Protein only microparticles with vaccine purposes or GFP-POMVs. As control, RPMI tissue culture media was also used for stimulation. As can be seen in FIG. 3, GFP-POMVs were able to stimulate the expression (detected in the supernatant) of a signature of proinflammatory and anti-inflammatory cytokines with statistical meaning. Lack of any deleterious effects observed in vitro and in vivo, confirms that the induction of non-toxic levels of these cytokines.

Data were obtained with a Luminex assay for the analysis in the supernatants of the macrophages of the following immune mediators: tumoral necrosis factor α (TNF α), Interleukin 1b (IL-1b), Interleukin 6 (IL-6), Interleukin 12 (IL-12), interferon γ (IFN-γ), interferon a (IFN-α), Interleukin 4 (IL-4) and Interleukin 10 (IL-10). From these data is deduced that the secretory granules induce first an unspecific response, which response is more complex of what was seen in in-vitro data (Example 2 in vitro with PBMC).

According to all these examples, in particular examples 2 and 3, it can be concluded that the immunogen of the invention, also herewith named POMVs or pp stimulates in vitro a balanced innate immune response that avoids excessive inflammation. The innate immune responses induced in vivo most probably provides the optimal environment for the induction of further adaptive immune responses (specific antibodies and T-cells); when the POMVs slowly de-granulate in the form of soluble nanoparticles and protein monomers (i.e., when the granule slowly disintegrated).

Example 4. Assay with mice. Study of the innate and adaptative response to the immunogenic composition of the invention

Animals: 12 eight weeks old BALB/c mice (6 males and 6 females separated in 2 cages) Inoculum: GFP protein-only microparticles (POMs), 50 or 5 ug/dose

Immunization regime: 2 immunizations, 2 weeks apart

Procedure: 6 mice (3 males and 3 females) were inoculated with 50 μg of GFP-H6 POMs and 6 mice (3 males and 3 females) were inoculated with 5 μg of GFP-H6 POMs. 3 animals from each group were euthanized 2 weeks after the second inoculation for the evaluation of the immunogenicity of GFP-H6 POMs. The 6 remaining animals were kept for 7 more weeks (a total of 9 weeks after the second inoculation) to analyze long-term immunogenicity

In vitro analyses:

### B. Anti-GFP Antibodies.

Indirect ELISA anti-soluble GFP-H6 (detection of mouse polyvalent Ig [G, A, M]). Coating with 5 μg/well of soluble GFP-H6.

### 2. GFP Specific T-Cell Responses.

FACS analysis of proliferating (Ki67+) cell subsets in mouse splenocytes, 9 weeks after the second inoculation and after in vitro stimulation for 5 days with GFP-H6 (soluble and

POMs, 5 μg/ml, and 50 μg/ml). Cells were stained with fluorescent anti-CD8 and anti-CD4 antibodies labeling the proliferating specific T-cell subsets.

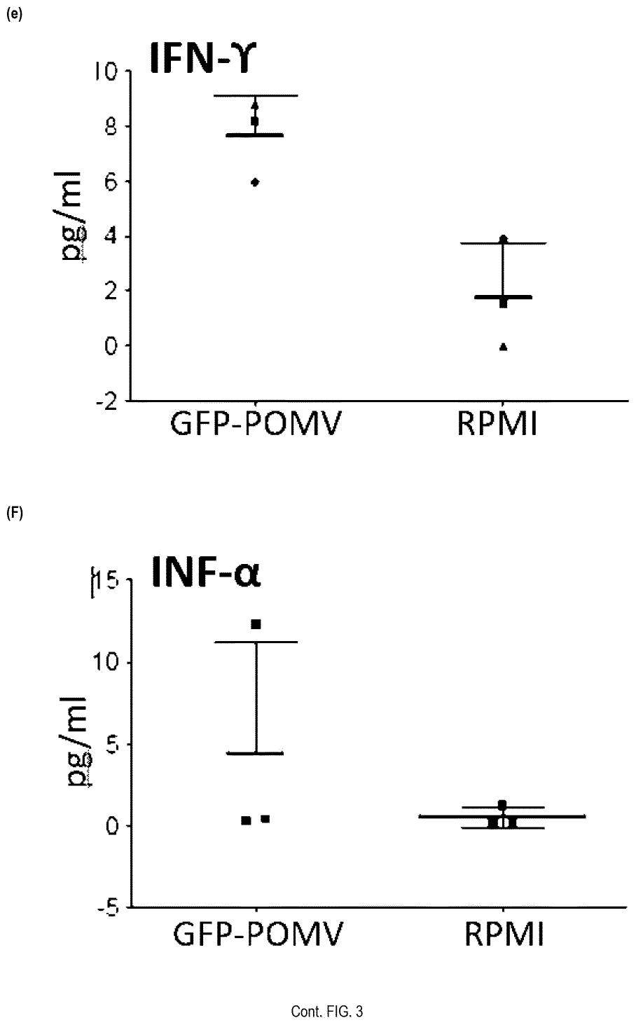

The results of the determined Anti-GFP antibodies induced after mouse immunization are depicted in FIGS. 5 (A) and (B). This figure illustrates that the microparticles of GFP (named P-POMVs or pp-GFP) induce GFP specific antibodies in mice in a dose-dependent manner. Moreover, the graphics at 2 (A) and 9 (B) weeks after immunization allow to affirm that the response is maintained along time, indeed 9 weeks after immunization. So, the circled data in both (A) and (B) illustrate the section of specific antibodies against GFP after immunizing mice with two doses of 50 μg of GFP-POMVs Non-immunized mice, (naïve), marks the background of the assay.

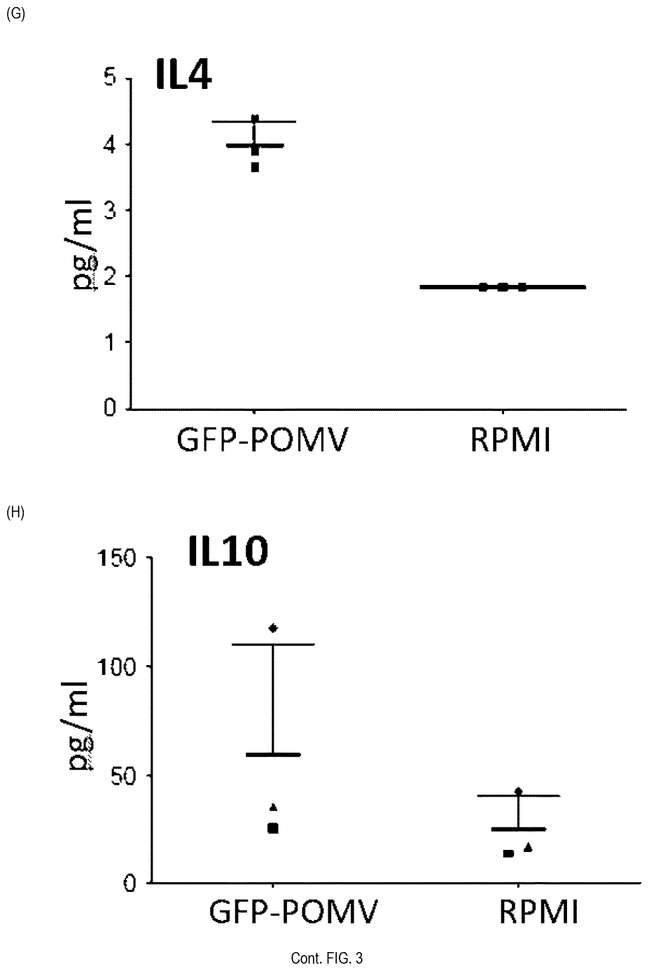

The results of the GFP specific T-cell responses are illustrated in FIG. 6.

Specific proliferation of CD8+ T-cells after the In vitro stimulation of the splenocytes obtained from mice immunized with the GFP-POMVs, but not from non-immunized control mice (naïve), clearly demonstrate their ability

These data allow concluding that the micropellets (GFP-POMVs in FIG. 6) induce specific CD8-T cells in mice, even at low concentrations (two doses of 5 micrograms). The ability of POMVs to induce CTL responses might be related to their micro or nanoparticulated nature, allowing to induce CD8 T-cell responses by cross-priming (ThijsW. H. Flinsenberg and Marianne Boes. Application of antigen cross-presentation research into patient care. Front. Immunol. 5:287. Doi: 10.3389/fimmu.2014.00287; Muntjewerff EM, Meesters LD and van den Bogaart G (2020) Antigen Cross-Presentation by Macrophages. Front. Immunol. 11:1276. Doi: 10.3389/fimmu.2020.01276), an ability that soluble proteins do not have.

The Induction of specific CD8-T cell responses is of high importance to obtain sterilizing immunity against intracellular pathogens (in its deffect, also tumoral cells). Thus, while antibodies can block the pathogens in fluids, CD8 T-cells are the only ones capable to specifically kill the infected cells destroying also the intracellular pathogen in them replicating. In fact most failures of subunit and inactivated vaccines obey to this lack of efficiency at the time of inducing CD8 T-cells

All these data, taken altogether, provide a new paradigm for vaccination with the capability of inducing a coordinate innate and adaptative response and, moreover, within the adaptative a whole antibody (B-cell) and T-cell response with the same immunogenic agent.

Data from examples 1 to 4 allow also concluding that the response is independent of the antigen assembled to conform the particle acting as immunogen, and also from the animal species to be vaccinated (data obtained from mice and pigs in these examples).

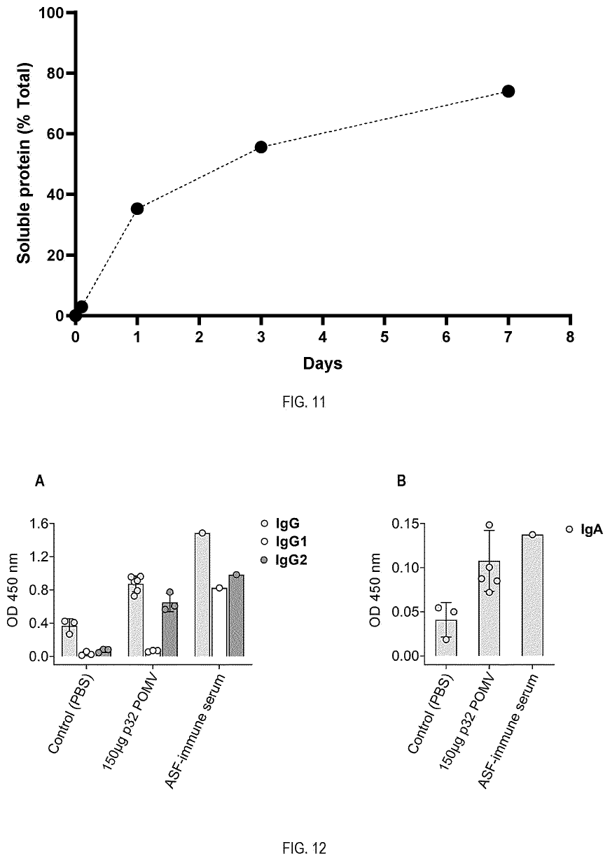

Example 5. Another experiment has confirmed not only the dose dependence of the responses induced by particles of the invention, but also that increasing the concentration of the microparticulated antigens, not only improved the specific IgG responses induced, but also stimulated detectable specific IgA responses both in mice and pigs (FIGS. 12 & 13) and independently of the antigen used (GFP and ASFVp32, respectively). Importantly, these responses were obtained in the absence of any additional adjuvant.

As described with lower doses of p32 secretion granules (p32-POMVS), prime-boosting of pigs with 150 μg of p32-POMVS induced better total IgG and IgG2a specific responses than any other formulation previously tested (FIG. 12A, and for the first time, specific IgA was found in blood at similar levels than that induced by our Live attenuated Vaccines (ASF-immune serum) (FIG. 12B).

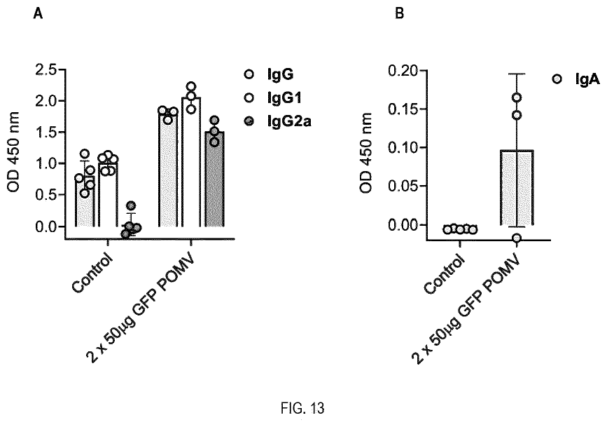

Interestingly, immunization with GFP secretion granules induced both IgG1 and IgG2a specific IgG isotypes (FIG. 13A), demonstrating that the secretion granule strategy has the ability to induce not only Th1 responses but also Th2 responses, depending on the nature of the encoded antigen. Similarly, anti-GFP IgA was also detected in mice when prime-boosting with 50 μg of GFP-POMVS (FIG. 13B), but not with 5 μg doses, confirming the dose dependence of the mucosal immune responses induced.

The Induction of specific IgAs after parenteral immunization open new expectation for using the secretory granules of the invention as a transversal platform to induce complete systemic and mucosal immunity in any animal species. The higher stability of microparticles compared to soluble proteins allows for an optimal behavior in the aggressive mucosal environments.

Further aspects/embodiments of the present invention can be found in the following clauses:

Clause 1.—Immunogenic composition comprising or consisting of a particle with a hydrodynamic diameter from 100 to 1500 nm, said particle comprising protein and/or peptide molecules and one or more salts of divalent cations, and wherein the protein and/or peptide molecules remain assembled in the presence of the said salts of divalent cations, being the ratio of moles of salt of divalent cation:moles of protein and/or peptide molecules in the particle from 4:1 to 1000:1.

Clause 2.—The immunogenic composition according to clause 1, wherein the particle has a release profile under physiologic conditions in phosphate buffered saline and agitation, in which an amount from 40% to 100% by weight in relation to the total weight of the proteins and/or peptide molecules is released with a time from 5 to 10 days in form of nanoparticles with a hydrodynamic diameter from 10 to 15 nm.

Clause 3.—The immunogenic composition according to any one of clauses 1-2, wherein the divalent cations are selected from the group consisting of Be²⁺, Mg²⁺, Mn²⁺, Ca²⁺, Sr²⁺, Ba²⁺, Ra²⁺Zn²⁺, Cu²⁺, Ni²⁺, and combinations thereof.

Clause 4.—The immunogenic composition according to any one of clauses 1-3, wherein the proteins and/or peptide molecules are selected from one or more antigenic proteins and/or peptides selected from the group consisting of a viral antigenic protein or peptide, a bacterial antigenic protein or peptide, a fungal antigenic protein or peptide, a protozoa antigenic protein or peptide, a cancer antigen, toxin antigen, venom antigen, autoimmune causing disease antigen, and a pathogenic antigen.

Clause 5.—The immunogenic composition according to clause 4, wherein the proteins and/or peptide molecules are selected from one or more antigenic proteins and/or peptides selected from the group consisting of a viral antigenic protein or peptide, a bacterial antigenic protein or peptide.

Clause 6.—The immunogenic composition according to clause 5, wherein the viral antigenic protein or peptide is one of an animal infecting virus, and the bacterial antigenic protein or peptide is one of an animal infecting bacteria.

Clause 7.—The immunogenic composition according to clause 6, the viral antigenic protein is protein p32 or a fragment thereof of African swine fever virus.

Clause 8.—A vaccine composition comprising a therapeutically effective amount of the immunogenic composition as defined in any one of clauses 1-7, together with a pharmaceutically and acceptable excipient and/or carrier.

Clause 9.—The vaccine according to clause 8, further comprising and adjuvant.

Clause 10.—The vaccine according to clause 9, wherein the adjuvant is selected the group consisting of alum, aluminium hydroxide, aluminium phosphate, Freund's complete adjuvant, squalene, monophosphoryl lipid A, the two-component liposomal adjuvant system CAF01, and combinations thereof.

Clause 11.—An immunogenic composition as defined in any one of clauses 1-7, or a vaccine as defined in any one of clauses 8-10 for use as a medicament.

Clause 12.—The immunogenic composition or the vaccine for use according to clause 11, which is for use in the prevention and/or treatment of diseases caused by a pathogen, in particular selected from a virus, a bacterium, a fungus, a protozoa and combinations thereof.

Clause 13.—A particle for use as immunogen, said particle with a hydrodynamic diameter from 100 to 1500 nm, said particle comprising protein and/or peptide molecules and one or more salts of divalent cations, and wherein the protein and/or peptide molecules remain assembled in the presence of the said salts of divalent cations, being the ratio of moles of salt of divalent cation:moles of protein and/or peptide molecules in the particle from 4:1 to 1000:1.

Clause 14.—A method for the preparation of a vaccine as defined in any of clauses 8-10, comprising the step of mixing the immunogenic composition of any of claims 1-7 with a pharmaceutically acceptable excipient and/or carrier.

Clause 15.—A vaccination kit comprising
(a) an immunogenic composition as defined in any of clauses 1-7;
(b) a pharmaceutically acceptable excipient and/or carrier;
(c) optionally an adjuvant; and
(d) optionally instructions for its use.

Moreover, still further aspects/embodiments of the present invention can be found in the following numbered embodiments:

1.—Immunogenic composition comprising or consisting of a particle with a hydrodynamic diameter from 50 to 4000 nm, said particle comprising protein and/or peptide molecules and one or more salts of divalent cations, and wherein the protein and/or peptide molecules remain assembled in the presence of the said salts of divalent cations, being the ratio of moles of salt of divalent cation:moles of protein and/or peptide molecules in the particle comprised from 4:1 to 1000:1.

2.—The immunogenic composition according to embodiment 1, which comprises or consists of a particle with a hydrodynamic diameter from 100 to 1500 nm, said particle comprising protein and/or peptide molecules and one or more salts of divalent cations, and wherein the protein and/or peptide molecules remain assembled in the presence of the said salts of divalent cations, being the ratio of moles of salt of divalent cation:moles of protein and/or peptide molecules in the particle comprised from 4:1 to 1000:1.

3.—The immunogenic composition according to any one of embodiments 1-2, wherein the particle has a release profile under physiologic conditions in phosphate buffered saline and agitation, in which an amount from 40% to 100% by weight in relation to the total weight of the proteins and/or peptide molecules is released within a time from 5 to 10 days in form of nanoparticles with a hydrodynamic diameter from 10 to 50 nm.

4.—The immunogenic composition according to any one of embodiments 1-3, wherein the divalent cations are selected from the group consisting of Be²⁺, Mg²⁺, Mn²⁺, Ca²⁺, Sr²⁺, Ba²⁺, Ra²⁺Zn²⁺, Cu²⁺, Ni²⁺, and combinations thereof.

5.—The immunogenic composition according to any one of embodiments 1-4, wherein the proteins and/or peptide molecules are selected from one or more antigenic proteins and/or peptides selected from the group consisting of a viral antigenic protein or peptide, a bacterial antigenic protein or peptide, a fungal antigenic protein or peptide, a protozoa or parasite antigenic protein or peptide, a cancer antigen, toxin antigen, venom antigen, autoimmune causing disease antigen, allergenic antigen, and a pathogenic antigen.

6.—The immunogenic composition according to embodiment 5, wherein the proteins and/or peptide molecules are selected from one or more antigenic proteins and/or peptides selected from the group consisting of a viral antigenic protein or peptide and a bacterial antigenic protein or peptide.

7.—The immunogenic composition according to embodiment 6, wherein the viral antigenic protein or peptide is one of an animal infecting virus, in particular, one of a virus selected from Influenza, Porcine reproductive and respiratory syndrome virus, and African swine fever virus, and the bacterial antigenic protein or peptide is one of an animal infecting bacteria, in particular one of a bacteria selected from Glasserela parasuis and Streptococcus suis.

8.—The immunogenic composition according to embodiment 7, the viral antigenic protein is protein p32 or a fragment thereof of African swine fever virus.

9.—The immunogenic composition according to any one of embodiments 1-8, which comprises or consists in particles with a hydrodynamic diameter from 50 to 4000 nm, said particles comprising:
protein and/or peptide molecules with one or more amino acids that due to the presence of charge at physiological pH and/or of the presence of aromatic or heteroaromatic structures coordinate with the divalent cations, in particular comprising polyhistidine-tags; and -salts of divalent cations; and wherein the protein and/or peptide molecules remain assembled in the presence of the said salts of divalent cations, being the ratio of moles of salt of divalent cation:moles of protein and/or peptide molecules in the particle from 4:1 to 1000:1.

10.—A vaccine composition comprising a therapeutically effective amount of the immunogenic composition as defined in any one of embodiments 1-9, together with a pharmaceutically and acceptable excipient and/or carrier.

11.—The vaccine according to embodiment 10, further comprising and adjuvant, in particular, an adjuvant selected the group consisting of alum, aluminium hydroxide, aluminium phosphate, Freund's complete adjuvant, squalene, monophosphoryl lipid A, the two-component liposomal adjuvant system CAF01, and combinations thereof.

12.—An immunogenic composition as defined in any one of embodiments 1-9, or a vaccine as defined in any one of embodiments 10-11 for use as a medicament.

13.—The immunogenic composition or the vaccine for use according to embodiment 12, which is for use in the prevention and/or treatment of diseases caused by a pathogen, in particular selected from a virus, a bacterium, a fungus, a protozoa and combinations thereof.

14.—A particle for use as immunogen, said particle with a hydrodynamic diameter from 50 to 4000 nm, in particular from 100 to 1500 nm, said particle comprising protein and/or peptide molecules and one or more salts of divalent cations, and wherein the protein and/or peptide molecules remain assembled in the presence of the said salts of divalent cations, being the ratio of moles of salt of divalent cation:moles of protein and/or peptide molecules in the particle from 4:1 to 1000:1.

15.—A method for the preparation of a vaccine as defined in any of embodiments 10-11, comprising the step of mixing the immunogenic composition of any of embodiments 1-9 with a pharmaceutically acceptable excipient and/or carrier.

### CITATION LIST

Snapper C M (2018) Distinct Immunologic Properties of Soluble Versus Particulate Antigens. Front. Immunol. 9:598. Doi: 0.3389/fimmu.2018.00598 EP2755680B1
ThijsW. H. Flinsenberg and Marianne Boes. Application of antigen cross-presentation research into patient care. Front. Immunol. 5:287. Doi: 10.3389/fimmu.2014.00287
Muntjewerff E M, Meesters L D and van den Bogaart G (2020) Antigen Cross-Presentation by Macrophages. Front. Immunol. 11:1276. Doi: 10.3389/fimmu.2020.01276
Monteagudo P L, Lacasta A, Lopez E, Bosch L, Collado J, Pina-Pedrero S, Correa-Fiz F, Accensi F, Navas M J, Vidal E, Bustos M J, Rodriguez J M, Gallei A, Nikolin V, Salas M L, Rodriguez F. BA71ΔCD2: a New Recombinant Live Attenuated African Swine Fever Virus with Cross-Protective Capabilities. J Virol. 2017 Oct. 13; 91(21):e01058-17
WO2020208065A1
López-Laguna et al., “In Vitro Fabrication of Microscale Secretory Granules”, Advanced Functional Materials Journal-2021, 210914, https://doi.org/10.1002/adfm.202100914

What is claimed is:

1-18. (canceled)

19. An immunogenic composition comprising a particle with a hydrodynamic diameter from 200 to 4000 nm, said particle comprising an antigenic protein and/or antigenic peptide and one or more salts of divalent cations, wherein:
(a) the particle maintains its hydrodynamic diameter after sonication, the sonication conditions being 5 rounds of 40 s, 0.5 pulse on and off, wave width of 10%, with a Branson sonifier 450, 3 mm-diameter titanium probe,
(b) the ratio of moles of salt of divalent cation:moles of antigenic protein and/or peptide in the particle is from 40:1 to 1000:1, and
(c) the particle has a release profile under physiologic conditions in phosphate buffered saline, in which an amount from 30% to 100% by weight in relation to the total weight of the antigenic protein and/or peptide is released within a time from 24 h to 10 days in form of nanoparticles with a hydrodynamic diameter from 5 to 80 nm.

20. The immunogenic composition according to claim 19, wherein the particle has a hydrodynamic diameter from 200 to 1500 nm.

21. The immunogenic composition according to claim 19, wherein the divalent cations are selected from the group consisting of Be²⁺, Mg²⁺, Mn²⁺, Ca²⁺, Sr²⁺, Ba²⁺, Ra²⁺, Zn²⁺, Cu²⁺, Ni²⁺, and combinations thereof.

22. The immunogenic composition according to claim 19, wherein the divalent cations comprise Zn²⁺.

23. The immunogenic composition according to claim 19, wherein the ratio of moles of salt of divalent cation:moles of antigenic protein and/or peptide in the particle is from 40:1 to 500:1

24. The immunogenic composition according to claim 19, wherein the assembled proteins and/or peptides contain one or more amino acids that due to the presence of charge at physiological pH and/or of the presence of aromatic or heteroaromatic structures coordinate with the divalent cations.

25. The immunogenic composition according to claim 24, wherein the assembled proteins and/or peptides contain a polyhistidine-tag.

26. The immunogenic composition according to claim 19, wherein the antigenic proteins and/or peptides are selected from the group consisting of a viral antigenic protein or peptide, a bacterial antigenic protein or peptide, a fungal antigenic protein or peptide, a protozoa or parasite antigenic protein or peptide, a cancer antigen, toxin antigen, venom antigen, autoimmune causing disease antigen, allergenic antigen, and a pathogenic antigen.

27. The immunogenic composition according to claim 26, wherein the antigenic proteins and/or peptides are selected from the group consisting of a viral antigenic protein or peptide and a bacterial antigenic protein or peptide.

28. The immunogenic composition according to claim 27, wherein the viral antigenic protein or peptide is one of an animal infecting virus, in particular, one of a virus selected from Influenza, Porcine reproductive and respiratory syndrome virus, and African swine fever virus, and the bacterial antigenic protein or peptide is one of an animal infecting bacteria, in particular one of a bacteria selected from Glasserela parasuis and Streptococcus suis.

29. The immunogenic composition according to claim 28, wherein the viral antigenic protein is protein p32 or an antigenic fragment thereof of African swine fever virus.

30. A vaccine composition comprising a therapeutically effective amount of the immunogenic composition as defined in claim 19, together with a pharmaceutically and acceptable excipient and/or carrier.

31. The vaccine according to claim 30, further comprising an adjuvant selected the group consisting of alum, aluminium hydroxide, aluminium phosphate, Freund's complete adjuvant, squalene, monophosphoryl lipid A, the two-component liposomal adjuvant system CAF01, and combinations thereof.

32. A method of treatment comprising administering to a subject in need thereof an immunogenic composition as defined in claim 29.

33. A method of treatment comprising administering to a subject in need thereof a vaccine as defined in claim 30.

34. A method for treating a disease caused by a pathogen, in particular a pathogen selected from a virus, a bacterium, a fungus, a protozoa and combinations thereof, said method comprising administering to a subject in need thereof an immunogenic composition as defined in claim 29.

35. A method for treating a disease caused by a pathogen, in particular a pathogen selected from a virus, a bacterium, a fungus, a protozoa and combinations thereof, said method comprising administering to a subject in need thereof a vaccine as defined in claim 30.

36. A method for inducing an innate and an adaptative immune response to an antigenic protein and/or antigenic peptide, said method comprising administering to a subject in need thereof a particle having a hydrodynamic diameter from 200 to 4000 nm, in particular from 100 to 1500 nm, and comprising the antigenic protein and/or peptide and one or more salts of divalent cations, wherein:
(a) the particle maintains its hydrodynamic diameter after sonication, the sonication conditions being 5 rounds of 40 s, 0.5 pulse on and off, wave width of 10%, with a Branson sonifier 450, 3 mm-diameter titanium probe,
(b) the ratio of moles of salt of divalent cation:moles of antigenic protein and/or peptide in the particle is from 40:1 to 1000:1, and
(c) the particle has a release profile under physiologic conditions in phosphate buffered saline, in which an amount from 30% to 100% by weight in relation to the total weight of the antigenic proteins and/or peptide is released within a time from 24 h to 10 days in form of nanoparticles with a hydrodynamic diameter from 5 to 80 nm.

37. A method for the preparation of a vaccine as defined in claim 30, said method comprising the step of mixing the immunogenic composition with a pharmaceutically acceptable excipient and/or carrier.

38. The method of treatment according to claim 32, which is for mucosal administration.

39. An immunogenic composition consisting of a particle with a hydrodynamic diameter from 200 to 4000 nm, said particle comprising an antigenic protein and/or antigenic peptide and one or more salts of divalent cations, wherein:
(a) the particle maintains its hydrodynamic diameter after sonication, the sonication conditions being 5 rounds of 40 s, 0.5 pulse on and off, wave width of 10%, with a Branson sonifier 450, 3 mm-diameter titanium probe,
(b) the ratio of moles of salt of divalent cation:moles of antigenic protein and/or peptide in the particle is from 40:1 to 1000:1, and
(c) the particle has a release profile under physiologic conditions in phosphate buffered saline, in which an amount from 30% to 100% by weight in relation to the total weight of the antigenic protein and/or peptide is released within a time from 24 h to 10 days in form of nanoparticles with a hydrodynamic diameter from 5 to 80 nm.


SEQUENCE LISTING (2 sequences)

SEQ ID NO: 1 - protein, 223 aa, synthetic construct
MRKRKRKRKGGSSRSSDFILNISMKMEVIFKTDLRSSSQVVFHAGSLYNWFSVEIINSGRIVTTAIKTLLSTVKYDIVKSARIYAGQGYTEHQAQEEWNMILHVLFEEETESSASSENIHEKNDNETNECTSSFETLFEQEPSSEVPKDSKLYMLAQKTVQHIEQYGKAPDFNKVIRAHNFIQTIYGTPLKEEEKEVVRLMVIKLLKKISFYLTYIKHHHHHH

SEQ ID NO: 2 - protein, 244 aa, synthetic construct
MSKGEELFTGVVPILVELDGDVNGHKFSVSGEGEGDATYGKLTLKFICTTGKLPVPWPTLVTTLTYGVQCFSRYPDHMKRHDFFKSAMPEGYVQERTISFKDDGNYKTRAEVKFEGDTLVNRIELKGIDFKEDGNILGHKLEYNYNSHNVYITADKQKNGIKANFKIRHNIEDGSVQLADHYQQNTPIGDGPVLLPDNHYLSTQSALSKDPNEKRDHMVLLEFVTAAGITHGMDELYKHHHHHH